(19) United States
(12) Patent Application Publication
(10) Pub. No.: US 2011/0040113 A1
(43) Pub. Date: Feb. 17, 2011

(54) Pure PEG-lipid conjugates

(76) Inventors: Nian Wu, North Brunswick, NJ (US); Brian Charles Keller, Antioch, CA (US)

Correspondence Address: Lee Pederson, 712 East Main Street, Sleepy Eye, MN, 56085, US

(21) Appl. No.: 12/802,197

(22) Filed: Jun. 1, 2010

Related U.S. Application Data
(60) Provisional application No. 61/217,627, filed on Jun. 2, 2009.
(60) Provisional application No. 61/284,065, filed on Dec. 12, 2009.

(51) Int. Cl. C07C 55/00; C07C 41/00

(52) U.S. Cl. 554/227; 568/679

(57) ABSTRACT

Syntheses of polyethyleneglycol (PEG)-lipid conjugates are disclosed. Such syntheses involve stepwise addition of small PEG oligomers to a glycerol backbone until the desired chain size is attained. Polymers resulting from the syntheses are highly monodisperse. The present invention provides several advantages such as simplified synthesis, high product yield and low cost for starting materials. The present synthesis method is suitable for preparing a wide range of conjugates.

In another aspect, the invention comprises PEG lipid conjugates having a glycerol backbone covalently attached to one or two monodisperse PEG chains and one or two lipids. These conjugates are especially useful for pharmaceutical formulations.

### CLAIM OF PRIORITY

This application claims priority to U.S. provisional patent application No. 61/217,627 entitled “PURE PEG-LIPID CONJUGATES” and filed on Jun. 2, 2009; and to U.S. provisional patent application No. 61/284,065 entitled “PURE PEG-LIPID CONJUGATES” and filed on Dec. 12, 2009.

### FIELD OF THE INVENTION

The present invention relates to syntheses of polyethyleneglycol (PEG)-lipid conjugates. More particularly, the invention relates to convenient and economic synthetic methods and compositions for preparing PEG-lipid conjugates with substantially monodisperse PEG chains.

### BACKGROUND OF THE INVENTION

When used as a delivery vehicle, PEG-lipid conjugates have the capacity to improve the pharmacology profile and solubility of lipophilic drugs. They also provide other potential advantages such as minimizing side effects and toxicities associated with therapeutic treatments.

Narrow molecular weight distribution of drug delivery polymers is crucially important for biomedical applications, especially if used for intravenous injections. For instance, PEG-8 Caprylic/Capric Glycerides are mixtures of monoesters, diesters, and triesters of glycerol and monoesters and diesters of polyethylene glycols with a mean relative molecular weight between 200 and 400. Partially due to allergic reactions observed in animals, the application of PEG-8 CCG for many water-insoluble drugs was restricted and a dose limit of approximately 6% of PEG-8 CCG was posted for human oral drug formulations.

With PEG chains produced from free radical polymerization, molecular weight distributions are not narrowly controlled for chains having molecular weights between about 200 and 1,200 daltons and above. Typically, far less than 50% of the polymers in a batch have exactly the targeted molecular weight. Narrower-distribution may be achieved with size exclusion chromatography, which can result in up to more of the PEG polymers having a targeted molecular weight. However it is extremely difficult to achieve a mono-distribution of purified PEGS.

Highly pure PEG chains with up to about 12 subunits are commercially available. However, these PEG's are extremely expensive and require additional synthetic steps to incorporate them into pharmaceutical and/or cosmetic formulations.

### BRIEF SUMMARY OF THE INVENTION

Syntheses of polyethyleneglycol (PEG)-lipid conjugates are disclosed. Such syntheses involve stepwise addition of small PEG oligomers to a glycerol backbone until the desired chain size is attained. Polymers resulting from the syntheses are highly monodisperse. The present invention provides several advantages such as simplified synthesis, high product yield and low cost for starting materials. The present synthesis method is suitable for preparing a wide range of conjugates.

In another aspect, the invention comprises PEG lipid conjugates having a glycerol backbone covalently attached to one or two monodisperse PEG chains and one or two lipids. These conjugates are especially useful for pharmaceutical formulations.

### BRIEF DESCRIPTION OF THE DRAWINGS

FIG. 1 depicts a LC-MS chromatogram of 1,2-dioleoyl-rac-3-monomethoxydodecaethylene glycol (mPEG-12)-glycerol

FIG. 2 depicts a mouse PK profile of itraconazole IV solutions.

FIG. 3 depicts a mouse PK profile of itraconazole oral solutions.

### ABBREVIATION LIST

The present invention is herein disclosed using the following chemical nomenclature:
DAG-PEGs: diacylglycerol-polyethyleneglycols
DMAP: N,N-dimethylamino pyridine
mPEG: monomethox polyethylene glycol ether
PEG 12: polyethyleneglycols 600
PEG 23: polyethyleneglycols 1000
PEG 27: polyethyleneglycols 1200
GDM-12: 1,2-dimyristoyl-rac-glycerol-3-dodecaethylene glycol
GDO-12: 1,2-dioleoyl-rac-glycerol-3-dodecaethylene glycol
GDC-12: 1,2-dicholoyl-rac-glycerol-3-dodecaethylene glycol
GDM-600: GDO-600: 1,3-dioleoyl-glycerol-2-dodecaethylene glycol
GDC-600: 1,3-dicholoyl-glycerol-2-dodecaethylene glycol
GDS-12: 1,2-distearoyl-rac-glycerol-3-dodecaethylene glycol
GOB-12: 1,2-bis(dodecaethylene glycol)glycerol-3-oleate
GMB-12: 1,2-bis(dodecaethylene glycol)glycerol-3-myristate
DSB-12: 1,2-bis(dodecaethylene glycol)glycerol-3-stearate
GOBH 1,2-bis(hexaethyle glycol)glycerol-3-oleate
GMBH 1,2-bis(hexaethyle glycol)glycerol-3-myristate
GCBH: 1,2-bis(hexaethyle glycol)glycerol 3-cholate
GCLBH: 1,2-bis(hexaethyle glycol)glycerol 3-cholesterol
GPBH: 1,2-bis(hexaethyle glycol)glycerol-3-palmitate
GDO-23: 1,2-dioleoyl-rac-glycerol-3-polyethylene (1000) glycol, n=23
GDO-27: 1,2-dioleoyl-rac-glycerol-3-polyethylene (1200) glycol, n=27
GDM-23: 1,2-dimyristoyl-rac-glycerol-3-polyethylene (1000) glycol, n=23
GDM-27: 1,2-dimyristoyl-rac-glycerol-3-polyethylene (1200) glycol, n=27
GDS-23: 1,2-distearoyl-rac-glycerol-3-polyethylene (1000) glycol, n=23
TPGS-VE: d-alpha-tocopheryl polyethylene glycol-1000 succinate
GDO-X-PEG 12: 1,2-dioleoyl-rac-glycerol-3-X-dodecaethylene glycol (“X” presents a linker/spacer, i.e., thiol, which can be found in the Table 3)
Cyclosporine: Cyclo[[(E)-(2S,3R,4R)-3-hydroxy-4-methyl-2-(methylamino)-6-octenoyl]-L-2-aminobutyryl-N-methylglycyl-N-methyl-Lleucyl-L-valyl-N-methyl-L-leucyl-L-alanyl-D-alanyl-N-methyl-L-leucyl-N-methyl-L-leucyl-Nmethyl -L-valyl]
POPC: palmitoyl-oleayl phosphatidylcholine

### DETAILED DESCRIPTION OF THE INVENTION

Embodiments of the present invention are described herein in the context of synthesis methods, intermediates, and compounds related to making PEG-lipid conjugates with narrowly defined molecular weights. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

10141 When employing PEG-lipid conjugates as drug delivery vehicles, it is becoming increasingly important to use well-characterized and highly pure conjugates. For example, U.S. Pat. No. 6,610,322, which is incorporated herein by reference, teaches that varying the length of PEG and acyl chains affects the packing parameters of the conjugates which in turn determine whether compositions of PEG-lipid conjugates form liposomes or not. In addition to affecting the physical structure of drug formulations, the choice of lipids and PEG sizes may have significant effects on pharmacokinetics and stability when formulating specific drug compounds with PEG-lipid conjugates. Therefore, uniform batches of conjugates having monodisperse PEG chains of a specific size are often highly preferable over batches having a range of PEG lengths.

The present invention provides high purity PEG-lipid conjugates having monodisperse PEG chains, and compounds and methods for the synthesis of these PEG-lipid conjugates starting with PEG oligomers of molecular weight ranging from about 110 to 300 daltons. The present invention also provides methods for the preparation of PEG-lipid conjugates including various lipids such as saturated or unsaturated fatty acids or bile acids. Such PEG-lipid conjugates can be used for drug delivery, especially for intravenous administration of poorly water soluble agents.

Generally, the invention includes compositions and methods for synthesizing PEG-lipid conjugates comprising a glycerol backbone with either one or two monodisperse PEG chains and either one or two lipids groups bonded to the backbone. Spacer or linker groups may be included between the backbone and the PEG chains and/or lipid groups.

Variations of the invention include glycerol backbones with two lipids and one monodisperse PEG chain (both isomers), glycerol backbones with one lipid and two monodisperse PEG chain (both isomers), and glycerol backbones with one lipid and one monodisperse PEG chain (all isomers) where the third position on the backbone may be a variety of compounds or moieties.

In addition, the invention provides methods to make pure 1,2 or 1,3 glycerol isomers. Commercially available 1,2 glycerol lipid diesters may be used to formulate many compounds by linking new moieties to the available position on the glycerol backbone. However, positional transformation occurs during the storage of these 1,2 glycerol diesters resulting in the formation of more stable 1,3 glycerol isomers, which may be present in fractions as great as about 30%. The present invention is the sole possiblity to produce and keep the enantiomer purity of 1,2 or 1,3 glycerol isomers. While the 1,2 or 1,3 isomers may sometimes be functionally equivalent, the choice of isomer may have implications in a variety of delivery process such as intracellular transport of lipophilic molecules as well as their use as vehicles in pharmaceutical applications. For example, isomers may differ in the ability to stabilize a compound during solubilizing and storage.

Conjugates having monodisperse PEG chains up to 1200 Daltons are useful for various drug delivery applications. Conjugates where PEG chains between about 300 and 600 daltons are especially useful for formulating liquid dosage forms such as for intravenous injection or oral solution. Conjugates where PEG chains between about 600 and 1,200 Daltons are especially useful for solid dosage forms such as capsules. A combination the above is useful for making a solid dosage form for poorly water soluble agents in which a liquid form of the above conjugates, typically with PEG chains between about 300 to 600 daltons, is used as a solvent and the solid form of the above conjugates, typically with PEG chains between about 600 to 1,200 Daltons, is used as a solidifier.

The present invention includes providing convenient and economical synthesis methods for preparing monodisperse PEG-lipid conjugates and provides various linear linkage groups for conjugating a lipid to a polymer. The present invention provides several advantages such as simplified synthesis, high product yield and low cost for starting materials since commercially available PEG oligomers are extremely expensive making their cost prohibitive for large scale production of similar PEG-lipid conjugates. In addition, the present synthesis method is preferable for preparing a wide range of PEG-spacer-lipid conjugates.

Synthesis of monodisperse PEG chains involves initially linking a short chain of PEG (having between 1 and 6 subunits) to a protected glycerol backbone. The PEG chain is lengthened by repeated etherification. An example is shown in Reaction Scheme 1.

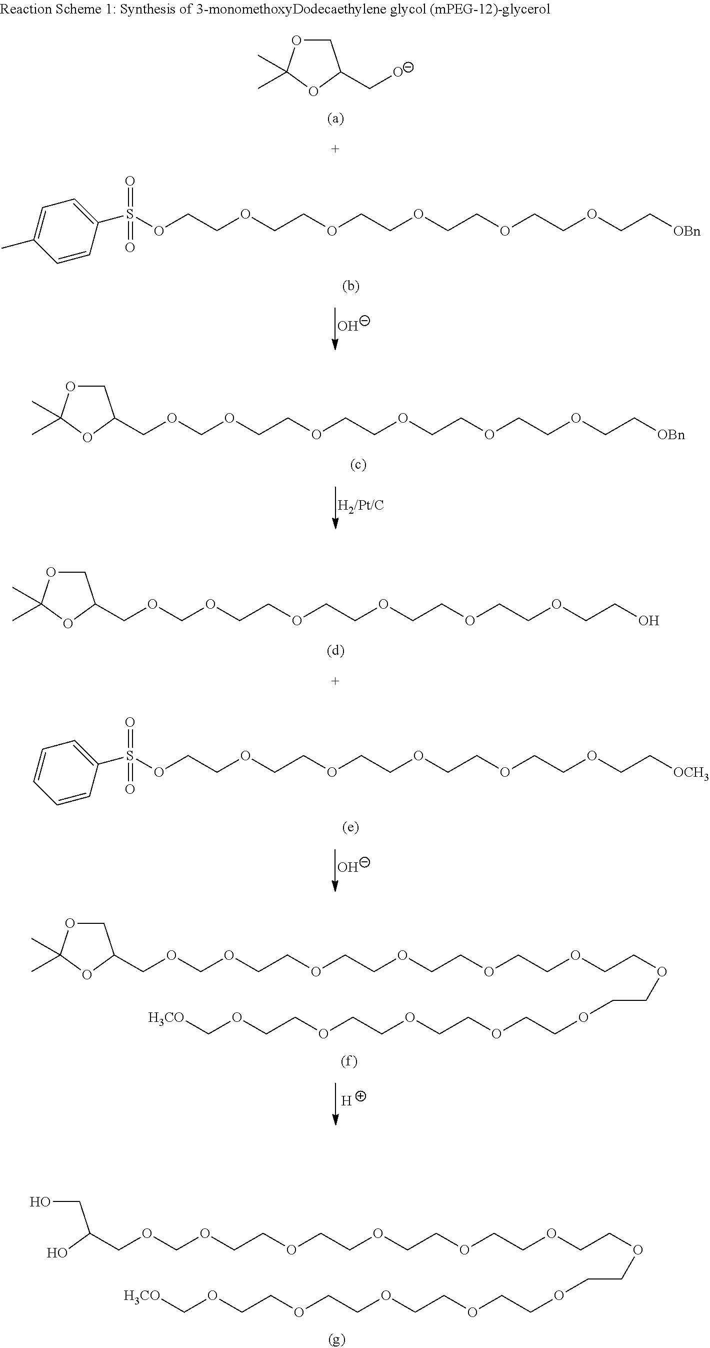

In Reaction Scheme 1 a first reactive PEG oligomer (b) is prepared by protecting (for example, by benzene) a first terminus of a PEG oligomer and creating a reactive second terminus (for example, by a tosyl group as shown). The first reactive oligomer is then combined with a glycerol that has two protected —OH groups (a). The protective group on the glycerol is selected to be stable under conditions that remove the protected group on the first terminus of the first reactive oligomer. The reactive second terminus of the oligomer bonds with the free —OH of the glycerol to form a glycerol-oligomer intermediate (c). The protecting group on the first terminus of the oligomer portion of the intermediate is then removed to expose a reactive —OH group (d). A second reactive PEG oligomer (e) is added to the intermediate to form an extended PEG chain attached to the glycerol backbone (f). In Reaction Scheme 1, the second reactive PEG oligomer is protected on its first terminus by a terminal methyl group, because a 12 subunit PEG chain is desired. If longer chains are desired, the protective group on the second reactive PEG oligomer is selected to that is can easily be removed for further extension of the PEG chain, for example by using (b) again as the second oligomer. Once the desired chain length is achieved, the protected groups of the glycerol backbone are removed to form the product (g). Product (g), having a monodisperse PEG chain, can then be further reacted to add desired lipids to the glycerol backbone. Similarity the synthesis can start with a short PEG chain or prepare the hexaethylene glycol from the etherification of two triethylene glycol or between a triethylene glycol and a monomethoxy triethylene glycol. In this route, two more steps will be involved in the synthesis.

In Reaction Scheme 1, removal of protective benzyl groups to expose a free hydroxyl group can be achieved by any suitable reagents. For example, the benzyl group can be removed by hydrogenation in presence of palladium catalyst before the PEG chain is extended by repeating the etherification process.

Following the synthesis of a PEG chain on a glycerol backbone as exemplified in Reaction Scheme 1, the protecting group is removed from glycerol, which results in 2 free hydroxyl groups. The free hydroxyl groups may be reacted with a fatty acid in the presence of N,N-dimethylamino pyridine (DMAP) in an inert solvent as shown below in Reaction Scheme 2.

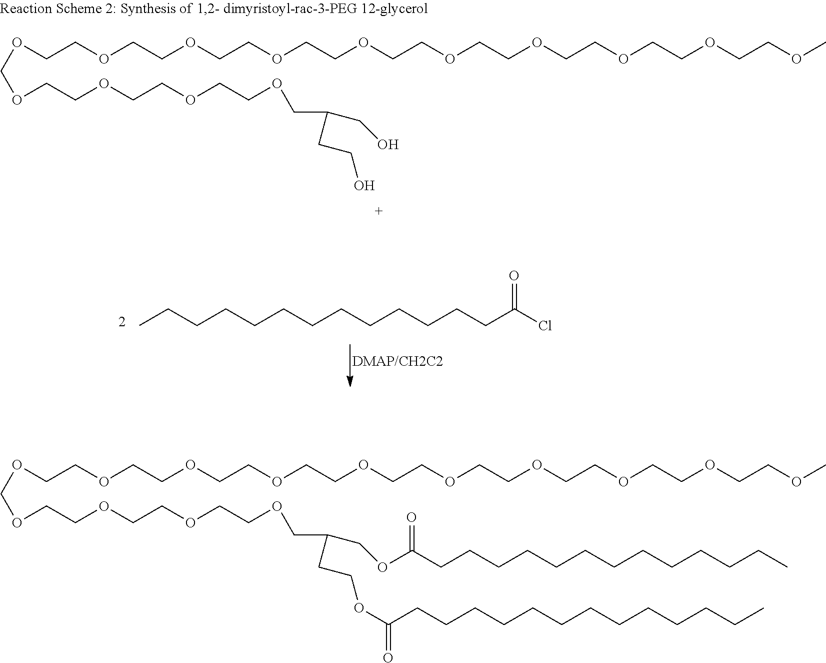

Reaction Scheme 3 depicts an approach to the preparation of an activated lipid to be used in Reaction Scheme 2. In this method, the carboxyl group of fatty acids is activated with a suitable activating agent. For example oxalyl chloride can be used as shown.

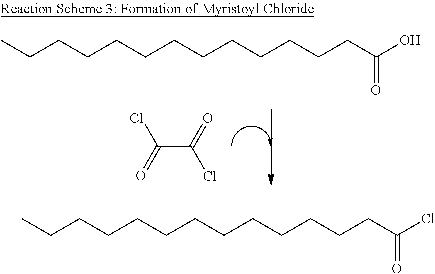

While the foregoing illustrates one method to synthesize a particular PEG-lipid conjugate having a single monodisperse PEG chain, the invention more broadly teaches methods and materials to make a wide range of PEG-lipid conjugates.

The first reactive PEG oligomer preferably comprises between 3 to 7 CH₂CH₂O units, and more preferably has 4 to 7 CH₂CH₂O units, though the oligomer may be of any length up to 12 units. Additional reactive oligomers also preferably comprise between 3 to 7 CH₂CH₂O units, and more preferably has 4 to 7 CH₂CH₂O units, though the additional oligomers may be of any length up to 12 units.

The PEG-lipid conjugates of the present invention each have one or two monodisperse PEG chains. Unless otherwise noted, more than 50% of the PEG chains in a particular conjugate have the same molecular weight. More preferably, more than 75% have the same molecular weight. Most preferably, more than 90% have the same molecular weight. Also unless otherwise noted, preferably the PEG chains are comprised of between about 6 and 27 polymer subunits. More preferably the PEG chains are comprised of between about 7 and 27 polymer subunits. Most preferably the PEG chains are comprised of between about 7 and 23 polymer subunits.

In the case of synthesizing 1,2-dimyristoyl-rac-3-PEG 12-glycerol, the glycerol is protected so that the PEG chain is formed on the 3 position. (see Reaction Scheme 1, compound (a)) It will be appreciated that employing alternate glycerol derivatives as starting components will result in conjugates having PEG chains in different positions. For example, protecting the 1 and 3 positions of the glycerol will result in a PEG chain at the 2 position (R). A glycerol derivative that may be used for such synthesis in shown in Chemical Structure 2.

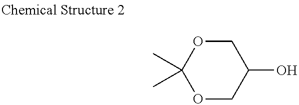

If a conjugate with two PEG chains is desired, glycerol derivatives as shown in Chemical Structure 3 or Chemical Structure 4 may be used. In these structures, R indicates either a protective group that may be replaced later, or an acyl lipid that may comprise the final structure. For these conjugates, the PEG chains are grown in tandem and will be identical in length. Conjugates having two PEG chains are particularly useful in some circumstances, as they function as branched PEG conjugates.

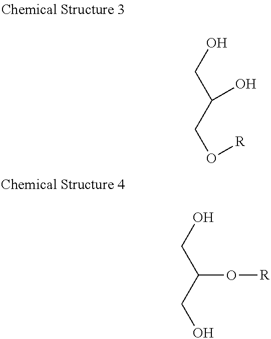

It may be desirable to incorporate linker groups other than oxyl between the glycerol backbone and the PEG chain(s). For example, a thiol linker may be employed for applications where a labile bond is useful. Other useful linkers are noted in Table 3 and elsewhere in this specification. For syntheses of conjugates having alternative linkers between the backbone and the PEG chain(s), the linker group is first attached to a protected glycerol backbone (e.g., Chemical Structure 3). Then the first reactive PEG oligomer is attached to the free end of the linker and the PEG is extended as desired. Alternatively, the first reactive PEG oligomer may be attached to the linker before bonding the linker to the backbone. In embodiments with linkers, preferred PEG-reagents have hydroxyl, amino, carboxyl, isocyanate, thiol, carbonate functional groups. Especially preferred PEG-reagents for use in this embodiment of the inventive method include PEG-tosylate, PEG-mesylate and succinyl-PEG.

It may be also be desirable to incorporate the same linker groups between the glycerol backbone and the lipid group(s). To obtain such conjugates, either the linker may be bonded with the lipid before attachment to the backbone, or the linker may be bonded to the backbone before attaching the lipid to the linker.

The foregoing approaches describe growing the PEG chain(s) on a backbone that is protected by a removable protecting group. Then, after the PEG is in place, the lipid group or groups are attached to the backbone. However, it is also possible to use one or two lipids as a protecting group or groups on the backbone before growing the PEG chain. This alternative approach is especially useful with alkyl chains that don't have reactive groups that need to be protected during PEG attachment and extension. It is much less useful when steroid acids conjugates are desired, as the bile acids tend to have many side groups that create issues during PEG attachment and extension.

While the synthetic methods described above are useful for making many compounds comprising the invention, in some cases it may be necessary or more convenient to employ other methods. For example, if a conjugate having a bile acid and two 27 subunit PEG chains is desired, such a conjugate may be constructed by synthesizing the monodisperse PEG chains before attaching them to the glycerol backbone. Similarly, it is possible to make many of the compounds of the invention including smaller PEGs by using PEG chains synthesized before attachment to the glycerol backbone.

Synthesis of other compounds of the invention may also require special considerations. Conjugates having linkers between the backbone and acyl groups or PEG sometimes will also preferably be made by building the monodisperse PEG chains before attaching them to the backbone, depending on considerations such as the nature of the bonds in the linkers.

Conjugates of the invention include those with a single lipid and a single monodisperse PEG chain attached to a glycerol backbone, where the third position on the backbone is occupied by another moiety ranging from a hydroxyl group to an active agent. It is worth noting that, while positional transformation occurs during the storage of 1,2 glycerol diesters having free hydroxyl groups as noted above, the chance of rearrangements will be much smaller for conjugates with a single lipid and a single monodisperse PEG chain attached to a glycerol backbone with a free hydroxyl group if the PEG chain is longer than about six subunits, since large energy is required to move a PEG chain (because the steric, molecular size and polarity are different than a lipid). Also, 1,3 isomers are generally more stable than 1,2 isomers.

Following the principles described above, a wide variety of PEG-lipid conjugates having one or two monodisperse PEG chains can be synthesized. A number of further specific embodiments are described hereinafter.

Suitable lipids for synthesis of PEG-lipid conjugates include bile acids (steroid acids) as well as alkyl chains. Therefore, the present invention includes a variety of PEG-lipid conjugates prepared by the present liquid phase synthesis method. The steroid acid-PEG conjugates can be incorporated into liposomes as a targeting moiety for lipid-based drug delivery to specific cells or as self-emulsifying drug delivery systems (SEDDS).

Bile acids (steroid acids) constitute a large family of molecules, composed of a steroid structure with four rings, a five or eight carbon side-chain terminating in a carboxylic acid, and the presence and orientation of different numbers of hydroxyl groups. The four rings are labeled from left to right A, B, C, and D, with the D-ring being smaller by one carbon than the other three. An exemplary bile acid is shown in Chemical Structure 5. All bile acids have side chains. When subtending a carboxyl group that can be amide-linked with taurine or glycine, the nuclear hydroxyl groups can be esterified with glucuronide or sulfate which are essential for the formation of water soluble bile salts from bile alcohols.

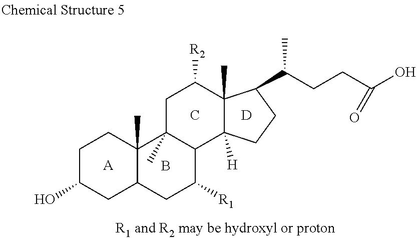

Currently only a few modifications in structure have been studied with respect to the physical-chemical properties of bile salts. One patent publication (WO 02083147) discloses bile salt fatty acid conjugate in which a bile acid or bile salt is conjugated in position 24 (carboxyl) with a suitable amino acid, and the unsaturated C═C bond is conjugated with one or two fatty acid radicals having 14-22 carbon atoms. That conjugate is intended to be used as a pharmaceutical composition for the reduction of cholesterol in blood, for the treatment of fatty liver, hyperglycemia and diabetes. Another patent (US 2003212051) discloses acyclovir-bile acid prodrugs in which a linker group may be used between the bile acid and the compound.

In one general embodiment, the present invention provides PEG-lipid conjugates according to general Formula I. The difference between the two variants shown in Formula I is the relative position of the polymer and lipid chains along the glycerol backbone.

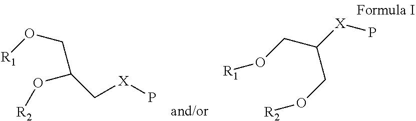

There are several alternative embodiments of Formula I. In one variation of Formula I, R1 and R2 may the same or different and are selected from the saturated and/or unsaturated alkyl groups listed in Table 1 or Table 2; X is —O—C(O)—, —O—, —S—, —NH—C(O)— or a linker selected from Table 3; and P is a PEG chain.

In another variation of Formula I, one of R1 and R2 is an alkyl group and the other is H. In these embodiments of Formula I, at least one of R1 or R2 is a saturated or unsaturated alkyl group having between 6 and 22 carbon atoms. In a preferred embodiment, R1 and R2 are the same and include between 6 and 22 carbon atoms and more preferably between 12 and 18 carbon atoms. The terms “alkyl” encompasses saturated or unsaturated fatty acids.

The present invention also provides PEG-lipid conjugates according to general formula II.

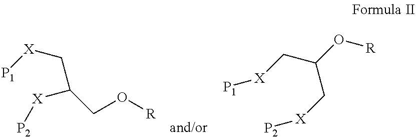

Again, there are several alternative embodiments of Formula II. In one variation of Formula II, R is an alkyl group listed in Tables 1 or Table 2; X is —O—C(O)—, —O—, —S—, —NH—C(O)— or a linker selected from Table 3; and P1 and P2 are the same PEG chains. By providing two branched PEG chains, conjugates according to Formula II may provide advantages over conjugates having a single longer PEG chain.

TABLE 1
Saturated lipids for use in the invention:

| | | | | Melting |
|---|---|---|---|---|
| Common | | | | point |
| name | IUPAC name | Chemical structure | Abbr. | (° C.) |
| Butyric | Butanoic acid | CH₃(CH₂)₂COOH | C4:0 | −8 |
| Caproic | Hexanoic acid | CH₃(CH₂)₄COOH | C6:0 | −3 |
| Caprylic | Octanoic acid | CH₃(CH₂)₆COOH | C8:0 | 16-17 |
| Capric | Decanoic acid | CH₃(CH₂)₈COOH | C10:0 | 31 |
| Lauric | Dodecanoic acid | CH₃(CH₂)₁₀COOH | C12:0 | 44-46 |
| Myristic | Tetradecanoic acid | CH₃(CH₂)₁₂COOH | C14:0 | 58.8 |
| Palmitic | Hexadecanoic acid | CH₃(CH₂)₁₄COOH | C16:0 | 63-64 |
| Stearic | Octadecanoic acid | CH₃(CH₂)₁₆COOH | C18:0 | 69.9 |
| Arachidic | Eicosanoic acid | CH₃(CH₂)₁₈COOH | C20:0 | 75.5 |
| Behenic | Docosanoic acid | CH₃(CH₂)₂₀COOH | C22:0 | 74-78 |

TABLE 2
Unsaturated lipids for use in the invention:

| | | Δ^{x} | |
|---|---|---|---|
| | | Location of | # carbon/ |
| Name | Chemical structure | double bond | double bonds |
| Myristoleic acid | CH₃(CH₂)₃CH═CH(CH₂)₇COOH | cis-Δ⁹ | 14:1 |
| Palmitoleic acid | CH₃(CH₂)₅CH═CH(CH₂)₇COOH | cis-Δ⁹ | 16:1 |
| Oleic acid | CH₃(CH₂)₇CH═CH(CH₂)₇COOH | cis-Δ⁹ | 18:1 |
| Linoleic acid | CH₃(CH₂)₄CH═CHCH₂CH═CH(CH₂)₇COOH | cis,cis-Δ⁹,Δ¹² | 18:2 |
| α-Linolenic acid | CH₃CH₂CH═CHCH₂CH═CHCH₂CH═CH(CH₂)₇COOH | cis,cis,cis- | 18:3 |
| | | Δ⁹,Δ¹²,Δ¹⁵ | |
| Arachidonic acid | CH₃(CH₂)₄CH═CHCH₂CH═CHCH₂CH═CHCH₂CH═CH(CH₂)₃COOH^{NIST} | cis,cis,cis,cis- | 20:4 |
| | | Δ⁵Δ⁸,Δ¹¹,Δ¹⁴ | |
| Erucic acid | CH₃(CH₂)₇CH═CH(CH₂)₁₁COOH | cis-Δ¹³ | 22:1 |

TABLE 3
Additional Linkers for use in the invention

| No | Symbol | Linker |
|---|---|---|
| 1 | N₁ | 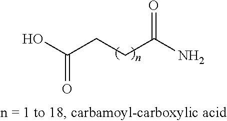 |
| 2 | N₂ | 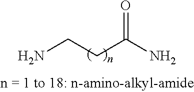 |
| 3 | N₃ | 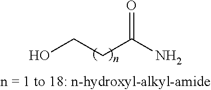 |
| 7 | N₇ | 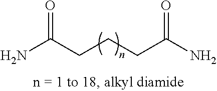 |
| 8 | N₈ | 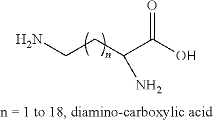 |
| 9 | N₉ | 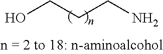 |
| 10 | N₁₀ | 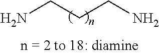 |
| 11 | N₁₁ | 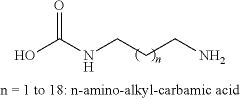 |
| 12 | N₁₂ | 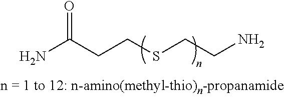 |
| 13 | S₁ | 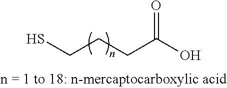 |
| 14 | S₂ | 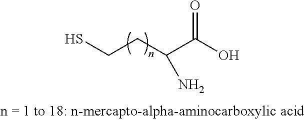 |
| 15 | S₃ | 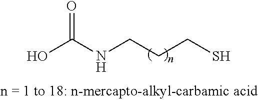 |
| 16 | S₄ | 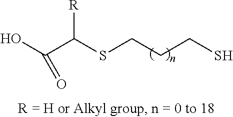 |
| 17 | S₅ | 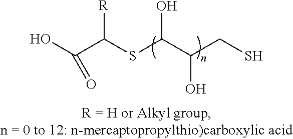 |
| 18 | S₆ | 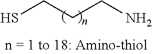 |
| 19 | S₇ | 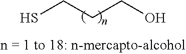 |
| 20 | S₈ | 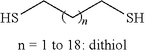 |
| 21 | S₉ | 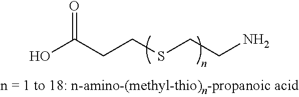 |
| 22 | Ac₁ | 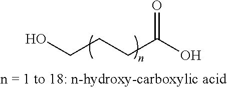 |
| 23 | Ac₂ | 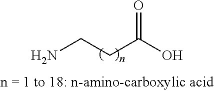 |
| 24 | Ac₃ | 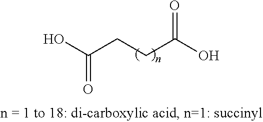 |
| 25 | Ac₄ | 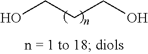 |
| 26 | Ac₅ | 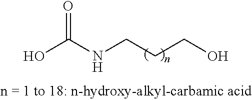 |
| 27 | Ac₆ | 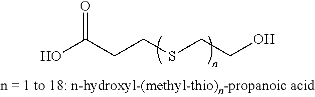 |

PEG-lipid conjugates of the present invention also include compounds where the lipid portion comprises one or two bile acids. These conjugates have the same structures as shown in Formula I and Formula II, except that the alkyl groups are replaced by bile acids. For bile acid conjugates, variations and preferred embodiments are the same as described for the PEG-alkyl conjugates. Because bile acids are similarly lipophilic to alkyl groups, bile acid conjugates also share similar physical properties and are generally suitable for some of the same uses as PEG-alkyl conjugates.

Chemical Structure 6 shows two variants of the present invention having a single PEG chain and two bile acids attached to a glycerol backbone.

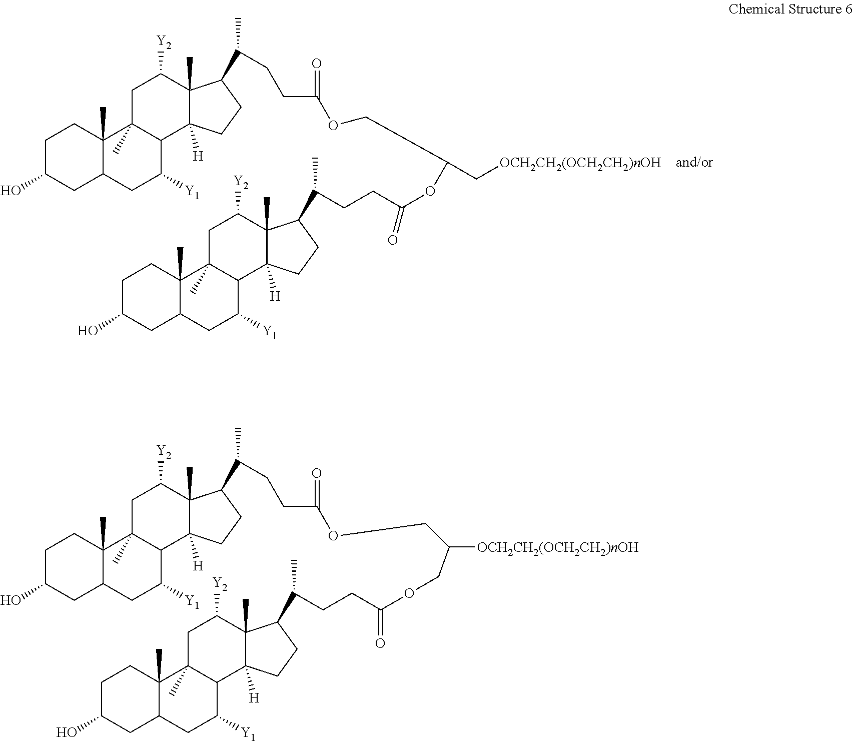

In Chemical Structure 6, Y1 and Y2 may be the same or different and are OH or H or CH₃, or are selected to accord with the bile acids shown in Table 4. Similarly, bile acids with differing side chains (as shown in Table 4) may be conjugated to the glycerol backbone. Table 4 lists bile acid and its derivatives that are useful in practicing the present invention.

TABLE 4
Bile acid (steroid acid) and its analogues for use in the Invention

| Name | Chemical Structure | Other Name |
|---|---|---|
| Cholic acid | 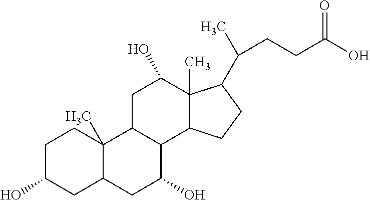 | 3α,7α,12α-trihydroxy- 5β-cholanoic acid |
| Desoxycholic acid | 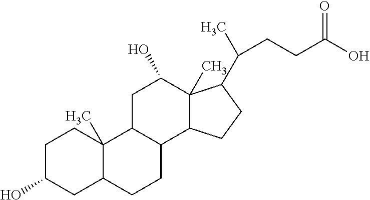 | 3α,12α-Dihydroxy-5β- cholanic acid |
| 5-Choleric acid-3β-ol | 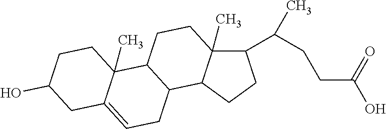 | 3β-Hydroxy-5-cholen-24- oic acid |
| Dehydrocholic acid | 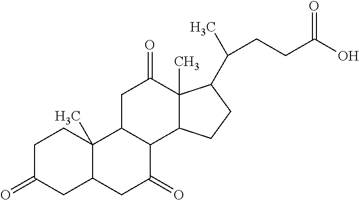 | 3,7,12-Trioxo-5β-cholanic acid |
| Glycocholic acid | 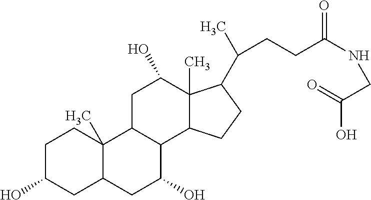 | N-(3α,7α,12α-Trihydroxy- 24-oxocholan-24-yl)- glycine |
| Glycodeoxycholic acid | 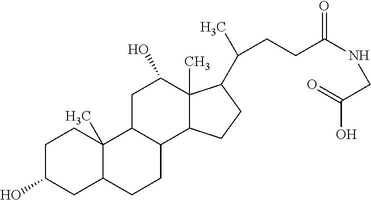 | N-(3α,12α-Dihydroxy-24- oxocholan-24-yl)glycine |
| Chenodeoxycholic acid | 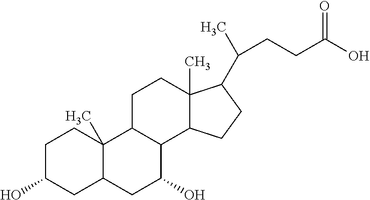 | 3α,7α-dihydroxy-5β- cholanic acid |
| Glycochenodeoxycholic acid | 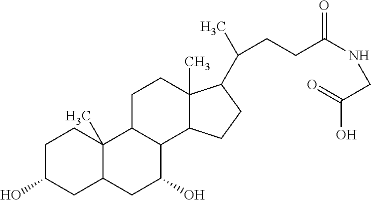 | N-(3α,7α-Dihydroxy-24- oxocholan-24-yl)glycine |
| Ursodeoxycholic acid | 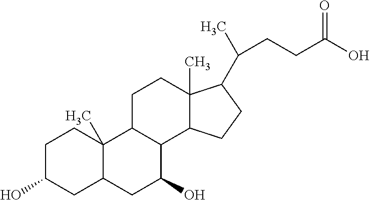 | Ursodiol |
| Lithocholic acid | 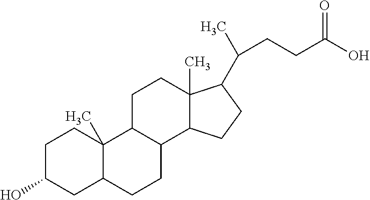 | 3α-Hydroxy-5β-cholan- 24-oic acid |
| Hyodeoxycholic acid | 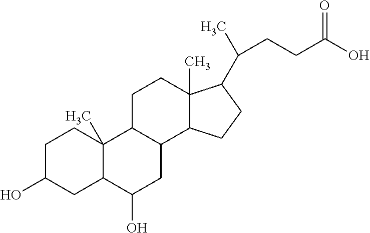 | 3α,6α-Dihydroxy-5β- cholan-24-oic acid |
| 5β-Cholanic acid-3,7-dione | 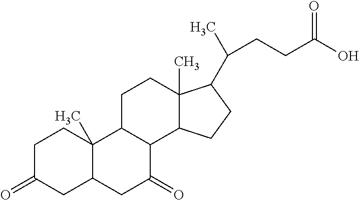 | 3,7-Diketo-5β-cholan-24- oic acid |

Yet another variation of the invention includes compounds accord to Formula I where either R1 or R2 is a bile acid and the other is an alkyl group. An example of this variation of lipid polymer conjugate is shown in Chemical Structure 7.

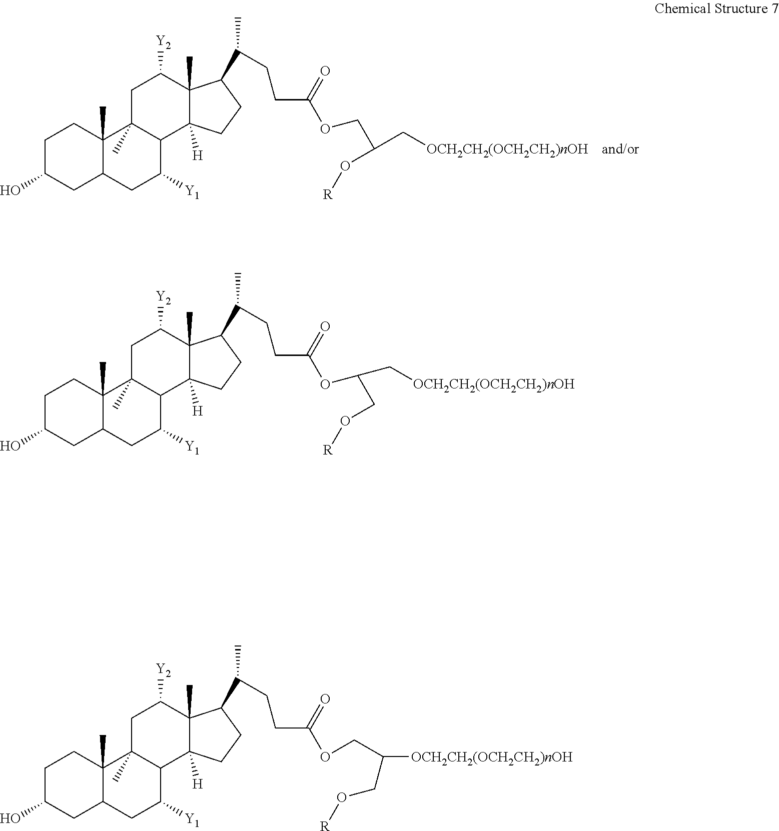

In Chemical Structure 3, Y1 and Y2 are the same or different and are OH or H or CH3 or selected in accord with the bile acids shown in Table 4. Also, the side chain of the bile acid may be varied according to the structures shown in Table 4. R is saturated and/or unsaturated alkyl group selected from Tables 1 and Table 2.

Another preferred embodiment for the compound of general Formula II is a PEG-bile acid conjugate according to Chemical Structure 8.

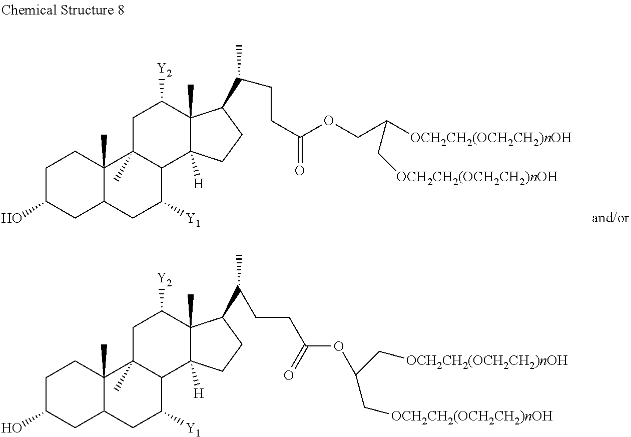

In Chemical Structure 8, Y1 and Y2 are OH or H or CH3 or selected according to the bile acids shown in Table 4. Also, the side chain of the bile acid may be varied according to the structures shown in Table 4.

Another further preferred embodiment for the compound of general Formula II is a PEG-cholesterol conjugate according to either of the structures shown in Chemical Structure 9.

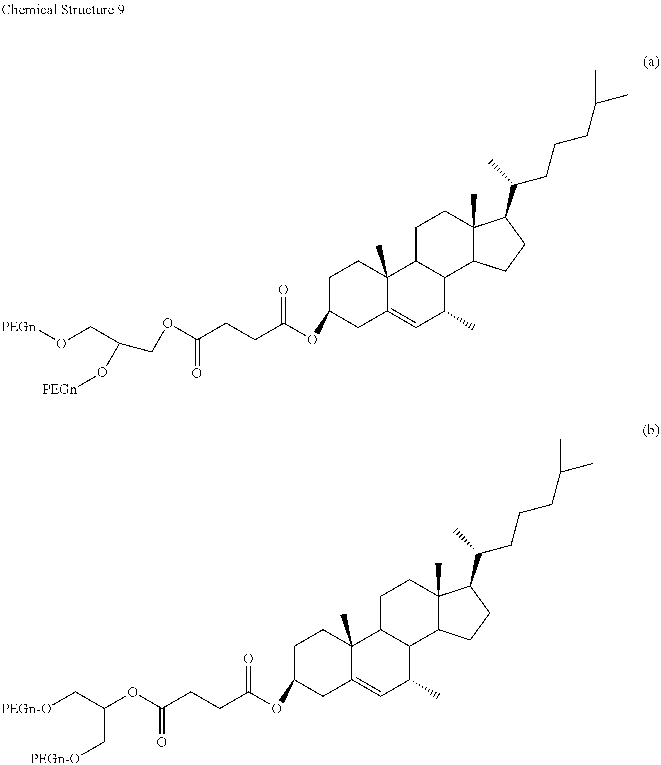

Another embodiment of the present invention is represented in Reaction Scheme 4. In this method, any suitable bile acid, such as cholic acid is reacted with 3-mPEG-12-glycerol in the presence of N,N-dimethylamino pyridine (DMAP) in dichloromethane to produce the final product of 1,2-dicholoyl-rac-3-mPEG 12-glycerol. It will be appreciated that monodisperse PEG chains of many discrete lengths may be used.

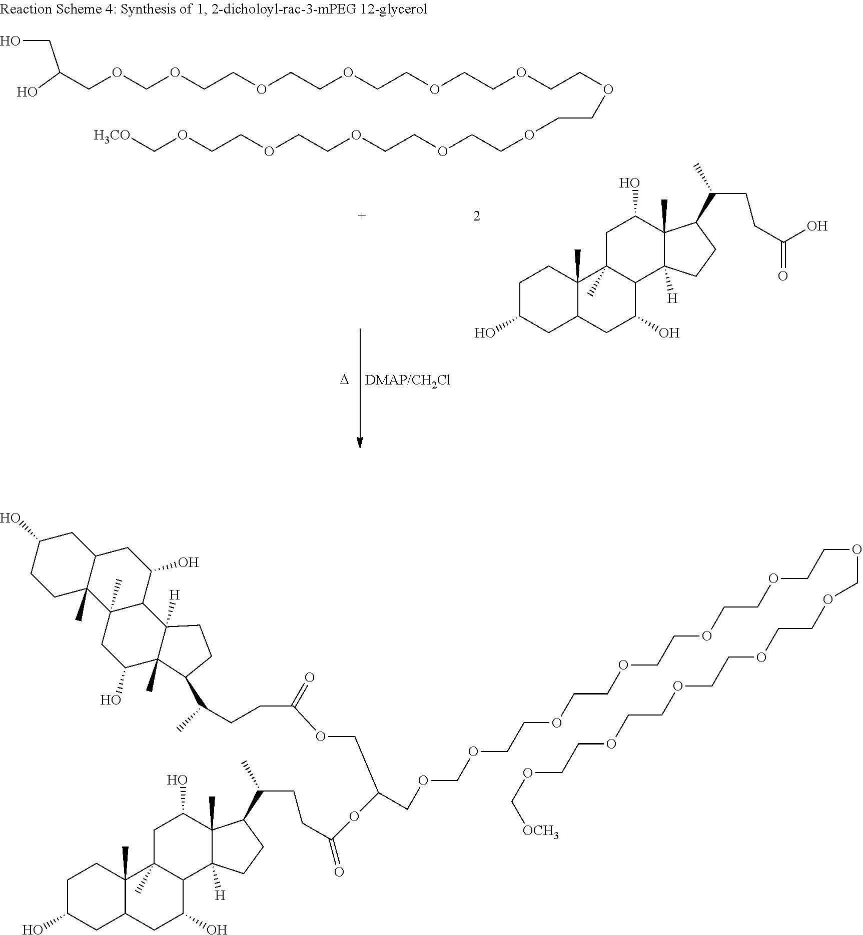

Another embodiment of the present invention, represented in Reaction Scheme 5, involves reaction of DL-1,2-isopropylideneglycerol intermediate with fatty acid to give I or with cholesterol to give II, respectively. Removal of ispropyl groups by any desired methods provides intermediate products III and IV respectively.

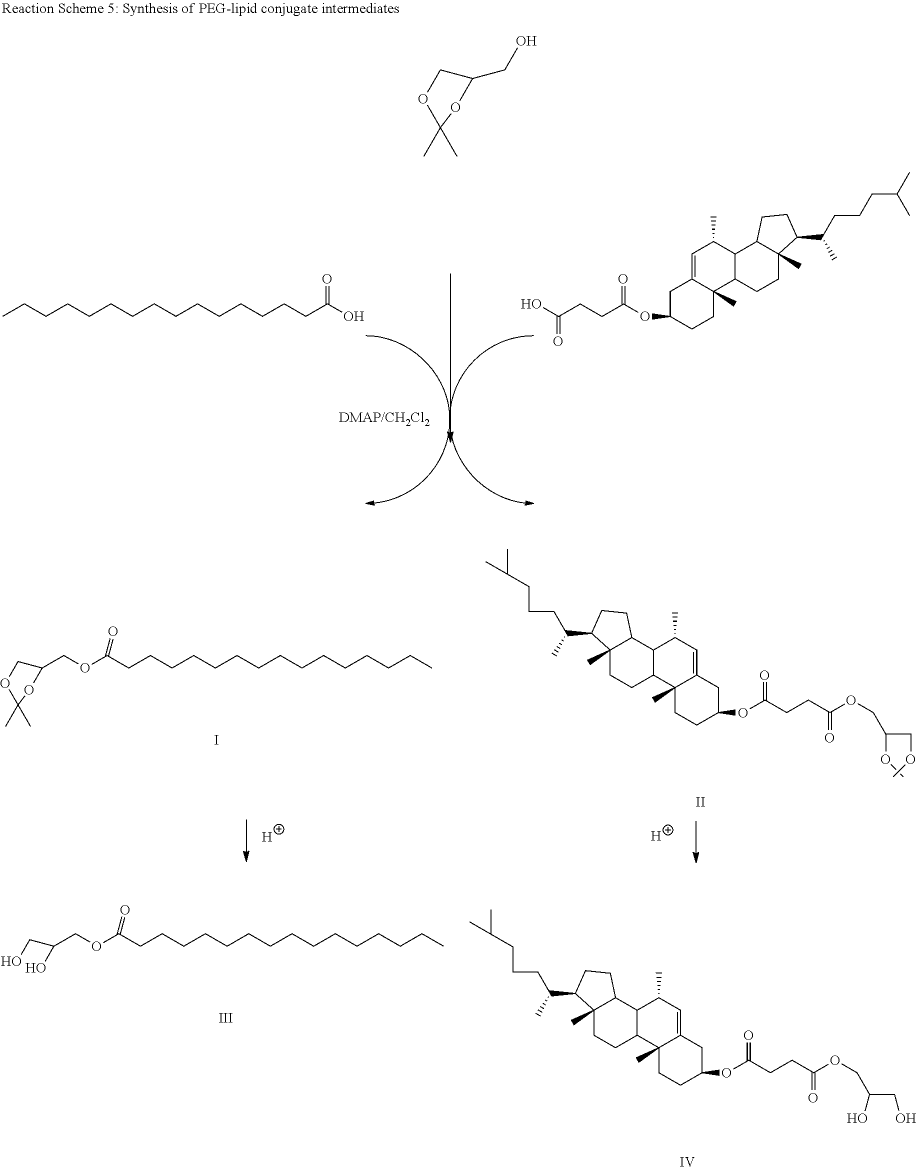

The described methods can be used to prepare a variety of novel PEG-lipid conjugates. For example, the methods can be used to prepare 3-PEG-1,2-alkylglycerol in pure form containing any fatty acid chain. Preferred fatty acids range from carbon chain lengths of about C6 to C22, preferably between about 10 and about C18.

The described methods can be used to prepare a variety of novel PEG-lipid conjugates. For example, the methods can be used to prepare 3-PEG-1,2-disteroid acid-glycerol in pure form containing any bile acid chain.

The described methods can be used to prepare a variety of novel branched PEG-lipid conjugates. For example, the methods can be used to prepare 3-alkylgl-1,2-bisPEG-gycerol in pure form containing any fatty acid chain. Preferred fatty acids range from carbon chain lengths of about C6 to C22, preferably between about C10 and about C18 (Reaction Scheme 6).

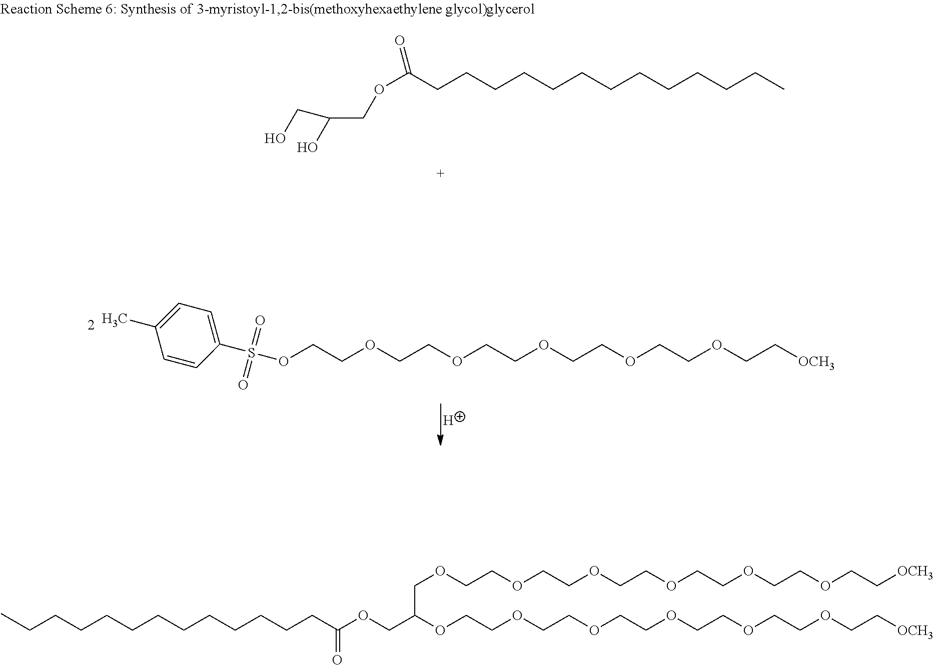

Reaction Scheme 6 results in a compound having a glycerol backbone, an lipid group, and two monodisperse PEG chains. However, it is worth noting that extending the PEG chain as exemplified in Reaction Scheme 1 can be done with other oligomers such as triethylene glycols or between triethylene glycol and monotriethylene glycol as described in the preceding section.

The described methods can be used to prepare a variety of novel branched PEG-lipid conjugates. For example, the methods can be used to prepare 3-steroid acid-1,2-bisPEG-gycerol in pure form containing steroid acid-glycerol in pure form containing any bile acid chain (Reaction Scheme 7).

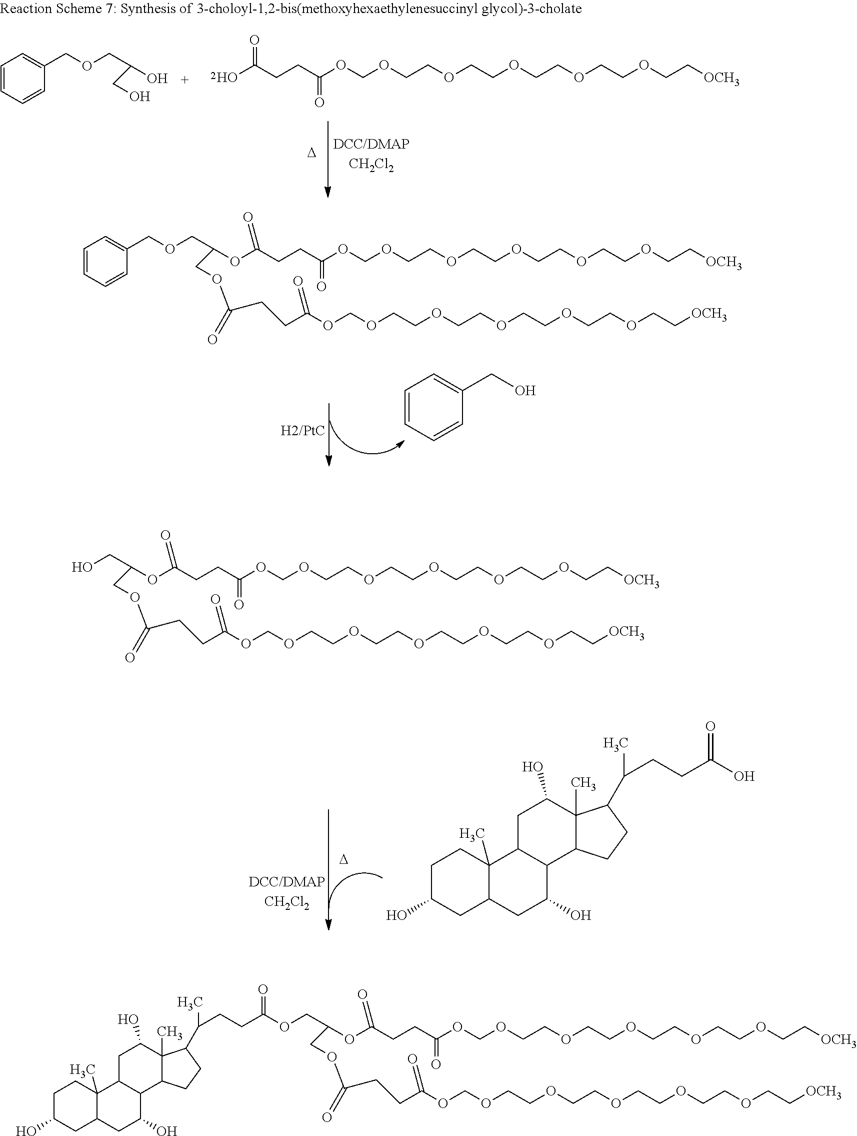

One preferred use for the inventive PEG-lipid is in the preparation of liposomes and other lipid-containing formulations. In accordance with the present invention, a pharmaceutical composition can include one or more genetic vectors, antisense molecules, proteins, peptides, bioactive lipids or drugs. For example, the active agent can include one or more drugs (such as one or more anticancer drugs or other anticancer agents). Typically hydrophilic active agents will be added directly to the formulation and hydrophobic active agents will be dissolved by PEG-lipid before mixing with the other ingredients.

Suitable active agents that can be present in the inventive formulation include one or more genetic vectors, antisense molecules, proteins, peptides, bioactive lipids or drugs, such as are described above. The inventive PEG-lipid can be used to administer active agents that are safer in presence of PEG oligomer for intravenous use.

Preferred active agents which are compatible with the present invention include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents can be selected from, for example, proteins, enzymes, hormones, nucleotides, polynucleotides, nucleoproteins, polysaccharides, glycoproteins, lipoproteins, polypeptides, steroids, terpenoids, retinoids, anti-ulcer H2 receptor antagonists, antiulcer drugs, hypocalcemic agents, moisturizers, cosmetics, etc. Active agents can be analgesics, anesthetics, anti-arrythmic agents, antibiotics, antiallergic agents, antifungal agents, anticancer agents (e.g., mitoxantrone, taxanes, paclitaxel, camptothecin, and camptothecin derivatives (e.g., SN-38), gemcitabine, anthacyclines, antisense oligonucleotides, antibodies, cytoxines, immunotoxins, etc.), antihypertensive agents (e.g., dihydropyridines, antidepressants, cox-2 inhibitors), anticoagulants, antidepressants, antidiabetic agents, anti-epilepsy agents, anti-inflammatory corticosteroids, agents for treating Alzheimers or Parkinson's disease, antiulcer agents, anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents, immunosuppressive agents, anti-gout agents, anti-malarials, anti-migraine agents, antimuscarinic agents, anti-inflammatory agents, such as agents for treating rheumatology, arthritis, psoriasis, inflammatory bowel disease, Crohn's disease, or agents for treating demyelinating diseases including multiple sclerosis, ophthalmic agents; vaccines (e.g., against influenza virus, pneumonia, hepatitis A, hepatitis B, hepatitis C, cholera toxin B-subunit, typhoid, plasmodium falciparum, diptheria, tetanus, herpes simplex virus, tuberculosis, HIV, bordetela pertusis, measles, mumps, rubella, bacterial toxoids, vaccinea virus, adenovirus, SARS virus, canary virus, bacillus calmette Guerin, klebsiella pneumonia vaccine, etc.), histamine receptor antagonists, hypnotics, kidney protective agents, lipid regulating agents, muscle relaxants, neuroleptics, neurotropic agents, opioid agonists and antagonists, parasympathomimetics, protease inhibitors, prostglandins, sedatives, sex hormones (e.g., androgens, estrogens, etc.), stimulants, sympathomimetics, vasodilators and xanthins and synthetic analogs of these species. The therapeutic agents can be nephrotoxic, such as cyclosporin and amphotericin B, or cardiotoxic, such as amphotericin B and paclitaxel. etopside, cytokines, ribozymes, interferons, oligonucleotides, siRNAs, RNAis and functional derivatives of the foregoing.

Chemotherapeutic agents are well suited for use in the inventive method. The inventive PEG-lipid formulations containing chemotherapeutic agents can be injected directly into the tumor tissue for delivery of the chemotherapeutic agent directly to cancer cells. In some cases, particularly after resection of a tumor, the liposome formulation can be implanted directly into the resulting cavity or can be applied to the remaining tissue as a coating.

The PEG-lipid in present invention can be used for preparing various dosage forms including tablets, capsules, pills, granules, suppositories, solutions, suspensions and emulsions, pastes, ointments, gels, creams, lotions, eye drop, powders and sprays in addition to suitable water-soluble or water-insoluble excipients.

The inventive PEG-lipid conjugates can be used to deliver the active agent to targeted cells in vivo. For example, the composition can be delivered orally, by injection (e.g., intravenously, subcutaneously, intramuscularly, parenterally, intraperitoneally, by direct injection into tumors or sites in need of treatment, etc.), by inhalation, by mucosal delivery, locally, and/or rectally or by such methods as are known or developed. Formulations containing PEGylated cardiolipin can also be administered topically, e.g., as a cream, skin ointment, dry skin softener, moisturizer, etc.

For in vivo use, the invention provides the use of a composition as herein described containing one or more active agents for preparing a medicament for the treatment of a disease. In other words, the invention provides a method of using a composition as herein described, containing one or more active agents, for treating a disease. Typically, the disease is present in a human or animal patient. In a preferred embodiment, the disease is cancer, in which instance, the inventive composition comprises one or more anticancer agents as active agents. For example, in accordance with the invention, the compositions as described herein can be employed alone or adjunctively with other treatments (e.g., chemotherapy or radiotherapy) to treat cancers such as those of the head, neck, brain, blood, breast, lung, pancreas, bone, spleen, bladder, prostate, testes, colon, kidney, ovary and skin. The compositions of the present invention, comprising one or more anticancer agents, are especially preferred for treating leukemias, such as acute leukemia (e.g., acute lymphocytic leukemia or acute myelocytic leukemia). Kaposi's sarcoma also can be treated using the compositions and methods of the present invention.

The following structures further illustrating the present invention.

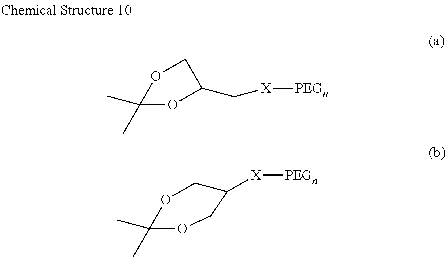

In Chemical Structure 10 “X” is a linker including oxy, thiol, amino, —COO—, —OCOO—, succinyl, haloid and those listed in Table 3. “n” is the number of repeating units. These structures represent intermediates in growing a single monodisperse PEG chain on a glycerol backbone, so n is generally between about 6 and 21. The PEG chain is extended through a sequential etherification starting with smaller chain such as triethylene glycol or tetraethylene glycol directly attached to the glycerol via a linker. The terminal group on the PEG chain may be, but is not limited to, a methyl group.

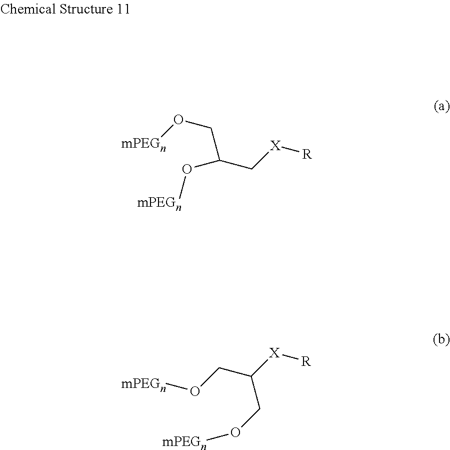

In Chemical Structure 11 “X” is the linker including oxy, thiol, amino, —COO—, —OCOO—, succinyl, haloid and those shown in Table 3. “n” is the number of repeating units. These structures represent the final step in growing two monodisperse PEG chains on a glycerol backbone. The “R” is an alkyl group such as saturated (Table 1) or unsaturated fatty acid (Table 2) or cholyl group or analog (Table 4). Terminal groups besides methyl may be included on the PEG chains.

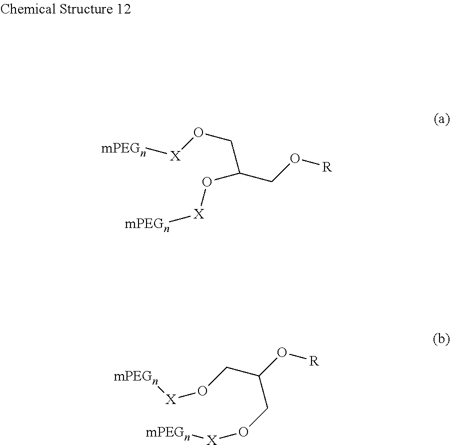

In Chemical Structure 12 “X” is the linker including oxy, thiol, amino, —COO—, —OCOO—, succinyl, haloid and alike and those shown in Table 3. “n” is the number of repeating units. These structures represent the final step in growing two monodisperse PEG chains on a glycerol backbone. Similarly the PEG chain is extended through a sequential etherification starting with smaller chain such as triethylene glycol or tetraethylene glycol directly attached to the glycerol via a linker. The “R” is an alkyl group such as saturated (Table 1) or unsaturated fatty acid (Table 2) or cholyl group or analog (Table 4). Terminal groups besides methyl may be included on the PEG chains.

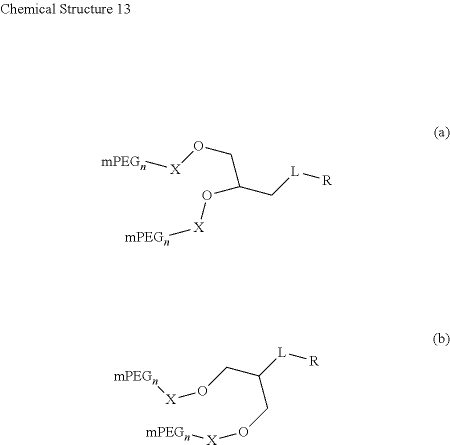

In Chemical Structure 13 “X” and “L” are the same or different linkers including oxy, thiol, amino, —COO—, —OCOO—, succinyl, haloid and those shown in Table 3. “n” is the number of repeating units. These structures represent the final step in growing two monodisperse PEG chains on a glycerol backbone, so n is generally between about 5 and 12. The “R” is an alkyl group such as saturated (Table 1) or unsaturated fatty acid (Table 2) or cholyl group and its analog (Table 4). Terminal groups besides methyl may be included on the PEG chains.

Embodiments of the present invention are described herein in the context of preparation of pharmaceutical compositions including purified PEG-lipid conjugates for increasing the solubility and enhancing the delivery of active agents. The approximate preferable compositions for formulated drug products are generally described herein, though different drugs typically have differing optimal formulations.

For IV solutions, the preferable concentration of drug is 0.1% to 30%. More preferable is 1 to 10%. Most preferable is 1 to 5%. The preferable ratio of PEG-lipid to the drug (PEG-Lipid/drug) is 1 to 20. More preferable is 1 to 10. Most preferable is 1 to 5.

For oral solutions, the preferable concentration of drug is 1% to 40%. More preferable is 2.5 to 30%. Most preferable is 5 to 30%. The preferable ratio of PEG-lipid to the drug (PEG-Lipid/drug) is 0.5 to 20. More preferable is 1 to 5. Most preferable is 1 to 3.

For ophthalmic preparations, the preferable concentration of drug is 0.01 to 5%. More preferable is 0.05 to 2%. Most preferable is 0.1 to 2%. The preferable ratio of PEG-lipid to the drug (PEG-Lipid/drug) is 1 to 20. More preferable is 3 to 15. Most preferable is 5 to 10.

For topical solutions, the preferable concentration of drug is 0.05 to 5%. More preferable is 0.1 to 5%. Most preferable is 0.1 to 2%. The preferable ratio of PEG-lipid to the drug (PEG-Lipid/drug) is 1 to 20. More preferable is 3 to 15. Most preferable is 5 to 10.

For oral capsules, the preferable capsule content of drug is 10 mg to 250 mg. More preferable is 25 mg to 200 mg. Most preferable is 25 mg to 100 mg. The preferable ratio of PEG-lipid to the drug (PEG-Lipid/drug) is 1 to 10. More preferable is 1 to 5. Most preferable is 2 to 5.

For topical preparations, the preferable concentration of drug is 0.05 to 5%. More preferable is 0.1 to 5%. Most preferable is 0.5 to 2%. The preferable ratio of PEG-lipid to the drug (PEG-Lipid/drug) is 1 to 50. More preferable is 3 to 20. Most preferable is 5 to 10.

While the foregoing discussion has focused on polymer-lipid conjugates having a glycerol backbone and including a PEG chains, the invention further includes alternate backbones and polymers. 3-amino-1, 2-propanediol, 3-bromo-1, 2-propanediol, 3-chloro-1, 2-propanediol, 3-fluoro-1, 2-propanediol, DL-glyceric acid, aspartic acid, glutamic acid and 1,2,4-butanetriol may be used as alternative backbones to synthesize similar PEG-lipid conjugates. Chemical Structure 14 illustrates a conjugate of the invention employing aspartic acid as a backbone. To prepare this conjugate, the starting material will be oleoyl alcohol instead of oleic acid since there are two carboxyl groups in the amino acid already. A succinate linker has been used to attach the PEG to the backbone. In such alternative embodiments, the PEG chain (or alternative polymer chain) is always monodisperse.

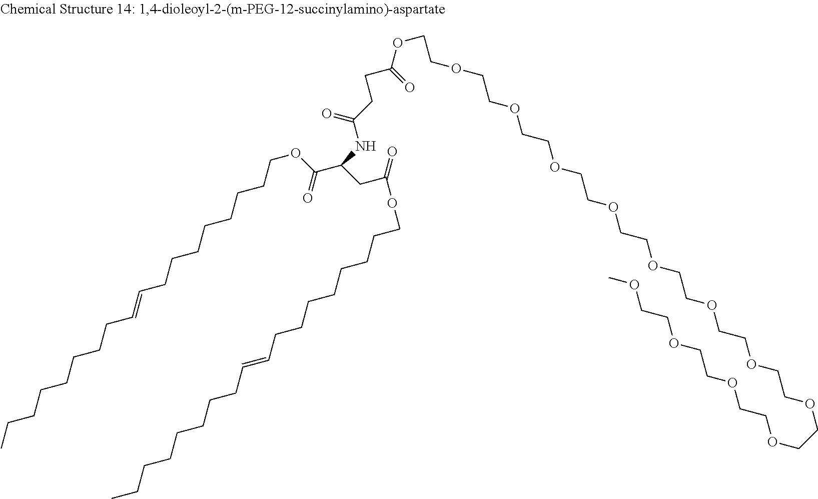

Propylene glycol and methylene glycol oligomers may be used as alternatives to ethylene glycol oligomers. Also, it is possible to create copolymers or block copolymers of these basic building blocks.

The synthetic methods described herein can be modified in any suitable manner. For example, the PEG-reagents for use in the inventive method can be any PEG derivative, which is capable of reacting with hydroxyl or amino group of central glycerol or 3-amino-1, 2-propanediol group or like or functional group of any linker.

The solvent for PEG-lipid conjugation reaction in the inventive method includes any solvent preferably a polar aprotic solvent such as N,N-dimethylformamide (DMF), dimethylsulfoxide (DMSO), pyridine, tetrahydrofuran (THF), dichloromethane, chloroform, 1,2-dichloroethane, dioxane and the like.

In one aspect, the invention is a method of making a PEG chain of a defined length, the method comprising (a) selecting a glycerol derivative with a glycerol protecting group that is stable under a first set of conditions and convertible to free hydroxyl groups under a second set of conditions; (b) selecting a initial PEG oligomer having between 1 and 12 subunits, where the initial PEG oligomer has an oligomer protecting group on its first terminus and the said oligomer protecting group converts to a hydroxyl group under the first set of conditions, and where the initial PEG oligomer has a reactive group on its second terminus, said reactive group forming a bond with a compound having a free hydroxyl group; (c) reacting the glycerol derivative with the initial PEG oligomer to form a glycerol-PEG conjugate; (d) removing the oligomer protective group by exposing the conjugate to the first set of conditions; (e) repeating steps (f), (g) and (h) between 0 and 6 additional times, where steps are as described below; (f) selecting an extending PEG oligomer having between 2 and 11 subunits, where the extending PEG oligomer has an oligomer protecting group on its first terminus and the said oligomer protecting group converts to a hydroxyl group under the first set of conditions, and where the extending PEG oligomer has a reactive group on its second terminus, said reactive group forming a bond with a compound having a free hydroxyl group; (g) reacting the glycerol-PEG conjugate with the extending PEG oligomer to form an extended glycerol-PEG conjugate; (h) removing the oligomer protective group by exposing the conjugate to the first set of conditions; (i) terminating the PEG chain by either step (j) or steps (k) and (l), where the steps are as described below; (j) adding a terminal group to the free hydroxyl group of the extended glycerol-PEG conjugate; or (k) selecting a terminal PEG oligomer having between 2 and 11 subunits, where the terminal PEG oligomer has terminal group on its first terminus, and where the terminal PEG oligomer has a reactive group on its second terminus, said reactive group forming a bond with a compound having a free hydroxyl group; and (l) reacting the glycerol-PEG conjugate or extended glycerol-PEG conjugate with the terminal PEG oligomer; and (m) exposing the terminated glycerol-PEG conjugate to the second set of conditions. The terminal group may be a methyl group. The first set of conditions may be catalytic reduction. The second set of conditions may be exposure to acid. The glycerol derivative may be a compound represented by the formula shown at Reaction Scheme 1(a). The glycerol derivative may be a compound represented by the formula shown as Chemical Structure 2. The glycerol derivative may be a compound represented by the formula shown as Chemical Structure 3. The glycerol derivative may be a compound represented by the formula shown as Chemical Structure 4. The glycerol protecting group may be an alkyl group. The method may further comprising the steps of: (n) removing the glycerol protecting group; and (o) bonding a lipid group to the glycerol backbone.

In another aspect, the invention is a chemical composition including a PEG-lipid conjugate, the PEG-lipid conjugate comprising: a glycerol backbone; a lipid group covalently attached to the glycerol backbone; and a PEG chain covalently attached to the glycerol backbone, where the PEG chain has a MW between about 200 and 1200 Daltons, and where greater than about 75 percent of the PEG chains of the conjugate molecules in the composition have the same MW. Greater than about 90 percent of the PEG chains of the conjugate molecules in the composition may have the same MW. The PEG chain may have a MW greater than about 600 Daltons. The lipid may be an alkyl group. The alkyl group may be selected from the alkyl groups in Table 1 and Table 2. The composition may further comprise a second lipid covalently attached to the glycerol backbone. The second lipid may be an alkyl group. The second alkyl group may selected from the alkyl groups in Table 1 and Table 2. The lipid may be a bile acid. The bile acid may be selected from the bile acids in Table 4. The bile acid may be cholesterol. The composition may further comprise a linker group between the glycerol backbone and the PEG chain. The linker may be selected from the group consisting of —S—, —O—, —N—, —OCOO—, and the linkers in Table 3. The composition may further comprise a second PEG chain covalently attached to the glycerol backbone. The linkage between the glycerol backbone and the second PEG chain may be selected from a group consisting of —O—C(O)—, —O—, —S—, and —NH—C(O)—. The linkage between the glycerol backbone and the second PEG chain may be selected from Table 3.

In another aspect, the invention include the compositions according to paragraph 089, where the glycerol backbone is replaced by a backbone selected from the group consisting of 3-amino-1, 2-propanediol, 3-bromo-1, 2-propanediol, 3-chloro-1, 2-propanediol, 3-fluoro-1, 2-propanediol, DL-glyceric acid, aspartic acid, glutamic acid, and 1,2,4-butanetriol.

In another aspect, the invention includes the compositions according to claim paragraph 089, where the PEG chains are replaced by polymers selected from the group consisting of polymethylene glycol, polypropylene glycol, and copolymers comprised of a at least two of the monomers selected from the group consisting of methylene glycol, propylene glycol and ethylene glycol.

In another aspect, the invention includes the following compounds: the compound represented by the formula shown at Reaction Scheme 1(a); the compound represented by the formula shown as Chemical Structure 2; the compound represented by the formula shown as Chemical Structure 3; the compound represented by the formula shown as Chemical Structure 4; the molecules of 1,2-isopropylidene-glycerol-3-ethylene glycol, 1,2-isopropylidene-glycerol-3-diethylene glycol, 1,2-isopropylidene-glycerol-3-triethylene glycol, 1,2-isopropylidene-glycerol-3-tetraethylene glycol, 1,2-isopropylidene-glycerol-3-pentaethylene glycol and 1,2-isopropylidene-glycerol-3-hexaethylene glycol, 1,2-isopropylidene-glycerol-3-heptaethylene glycol and 1,2-isopropylidene-glycerol-3-octaethylene glycol; and the molecules of 1,3-diacylglycerol-2-ethylene glycol, 1,3-diacylglycerol-2-diethylene glycol, 1,3-diacylglycerol-2-triethylene glycol, 1,3-diacylglycerol-2-tetraethylene glycol, 1,3-diacylglycerol-2-pentaethylene glycol, 1,3-diacylglycerol-2-hexaethylene glycol, 1,3-diacylglycerol-2-heptaethylene glycol and 1,3-diacylglycerol-2-octaethylene glycol.

In another aspect, the invention includes a method for increasing the bioavailability and/or solubility of an active agent, said method comprising: formulating the active agent with one or more of the a PEG-lipid conjugates of the present invention and administering said PEG-lipid conjugate based formulation to an animal or human.

In another aspect, the invention includes a chemical compound having the formula:

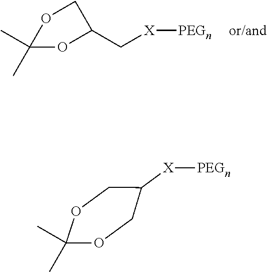

where n is between about 7 and 12; and where X is a linker group. X may have a MW between about 16 and 200. X may be selected from the group consisting of oxy, thiol, amino, —COO—, —OCOO—, succinyl, haloid and linkers shown in Table 3. The terminus of the PEG chain may have a MW between about 15 and 210. The terminus of the PEG chain may be a methyl group. The terminus of the PEG chain may be a protecting group. The terminus of the PEG chain may be a hydroxyl group.

In another aspect, the invention includes a chemical compound having the formula:

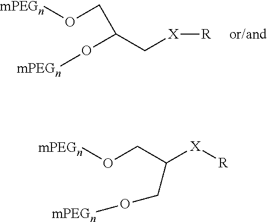

where n is between about 3 and 23; R is a lipid; and where X is a linker group. X may have a MW between about 14 and 620. X may be selected from the group consisting of oxy, thiol, amino, —COO—, —OCOO—, succinyl, haloid and linkers shown in Table 3. n may be between about 4 and 12. More preferably, n may be between about 7 and 12. The terminus of the PEG chain may have a MW between about 15 and 210. The terminus of the PEG chain may be a methyl group. R may be an alkyl group selected from Table 1 or Table 2. R may be a bile acid. R may be a bile acid selected from Table 4. R may be cholesterol.

In another aspect, the invention includes a chemical compound having the formula:

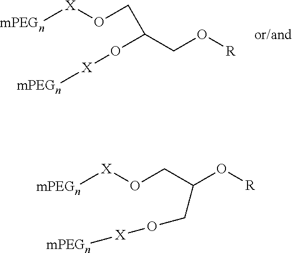

where n is between about 3 and 23; R is a lipid; R is a lipid; and where X are the same or different linker groups. X may have a MW between about 14 and 620. X may be selected from the group consisting of oxy, thiol, amino, —COO—, —OCOO—, succinyl, haloid and linkers shown in Table 3. n may be between about 4 and 23. n is preferably between about 7 and 23. The terminus of the PEG chain may have a MW between about 15 and 210. The terminus of the PEG chain may be a methyl group. R may be an alkyl group selected from Table 3 or Table 4. R may be a bile acid. R may be selected from Table 4. R may be cholesterol.

In another aspect, the invention includes a chemical compound having the formula

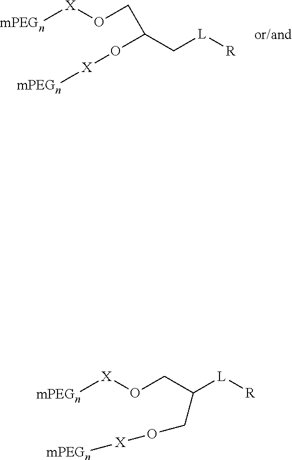

where is between about 3 and 23; R is a lipid; R is a lipid; L is a linker group; and where X are the same or different linker groups. X may have a MW between about 14 and 620. X may be selected from the group consisting of oxy, thiol, amino, —COO—, —OCOO—, succinyl, haloid and linkers shown in Table 3. n may be between about 4 and 23. n may be between about 7 and 23. The termini of the PEG chains may have a MW between about 15 and 210. The termini of the PEG chains may be methyl groups. R may be an alkyl group selected from Table 1 or Table 2. R may b a bile acid. R may be selected from Table 4. R may be cholesterol. X may b selected from the group consisting of oxy, thiol, amino, —COO—, —OCOO—, succinyl, haloid and linkers shown in Table 3.

### Examples

The following examples are further illustrating the invention and should not be constructed as in any way limiting its scope.

### Example 1

### Synthesis of 3-Oleoyl-1,2-bis(methoxyhexathylene glycol)glycerol

### Part 1A: 3-Benzyl-1,2-bis(methoxyhexathylene glycol)glycerol

To a three-necked flask, (±)-3-Benzyloxy-1,2-propanediol (1.2 g, 6 mmol), NaH (0.96 g, 40 mmol) and dry THF (150 mL) were added. A dry THF solution (50 mL) of monomethoxyhexaethylene glycol tosylate (5.4 g,12 mmol) was then added to the mixture dropwise at room temperature. The mixture was refluxed for 24 hours and cooled to room temperature. Ice-cold methanol was added to the reaction mixture to quench excessive NaH. The solvent was evaporated and the crude product was extracted with 5% HCl (w/v) and CH₂Cl₂. The solvent was evaporated and further purified by gel permeation chromatography to yield 85% of colorless liquid.

### Part 1B: 3-hydroxyl-1,2-bis(methoxyhexaethylene glycol)glycerol

To a solution of 5 grams of 3-Benzyl-1,2-bis(methoxyhexaethylene glycol)glycerol in 20 mL of n-Hexane, 5 drops of acetic acid and 0.6 g of palladium black were added. The mixture was purged with pure hydrogen at 30° C. in atmosphere for approximately 60 minutes to remove the benzyl protection group on the 3′-hydroxy. After the hydrogen was replaced by nitrogen, the solution was cooled to 4 to 6° C. and the catalyst was removed by filtration. Solvent was evaporated to yield 98% of the final product.

### Part 1C: 3-Oleoyl-1,2-bis(methoxyhexaethylene glycol)glycerol

6.5 g of the product from 1B (10 mmoles), 3.1 g of oleic acid (11 mmoles), 9.6 g of N,N′-Dicyclohexylcarbodiimide (50 mmol) and a catalytic amount of DMAP (0.6 g, 5 mmoles) in anhydrous CH₂Cl₂ (400 mL) was stirred at 25° C. for 12 h under nitrogen, after which the N,N′-dicyclohexylurea salts were precipitated and removed by filtration. The filtrates were evaporated under reduced pressure to yield 89% of the final product shown by Chemical Structure 15.

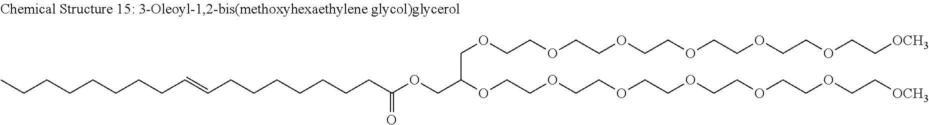

### Example 2

### Synthesis of 1,2-dioleoyl-rac-3-monomethoxydodecaethylene glycol (mPEG-12)-glycerol

The general steps for this synthesis are showed in Reaction Scheme 8.

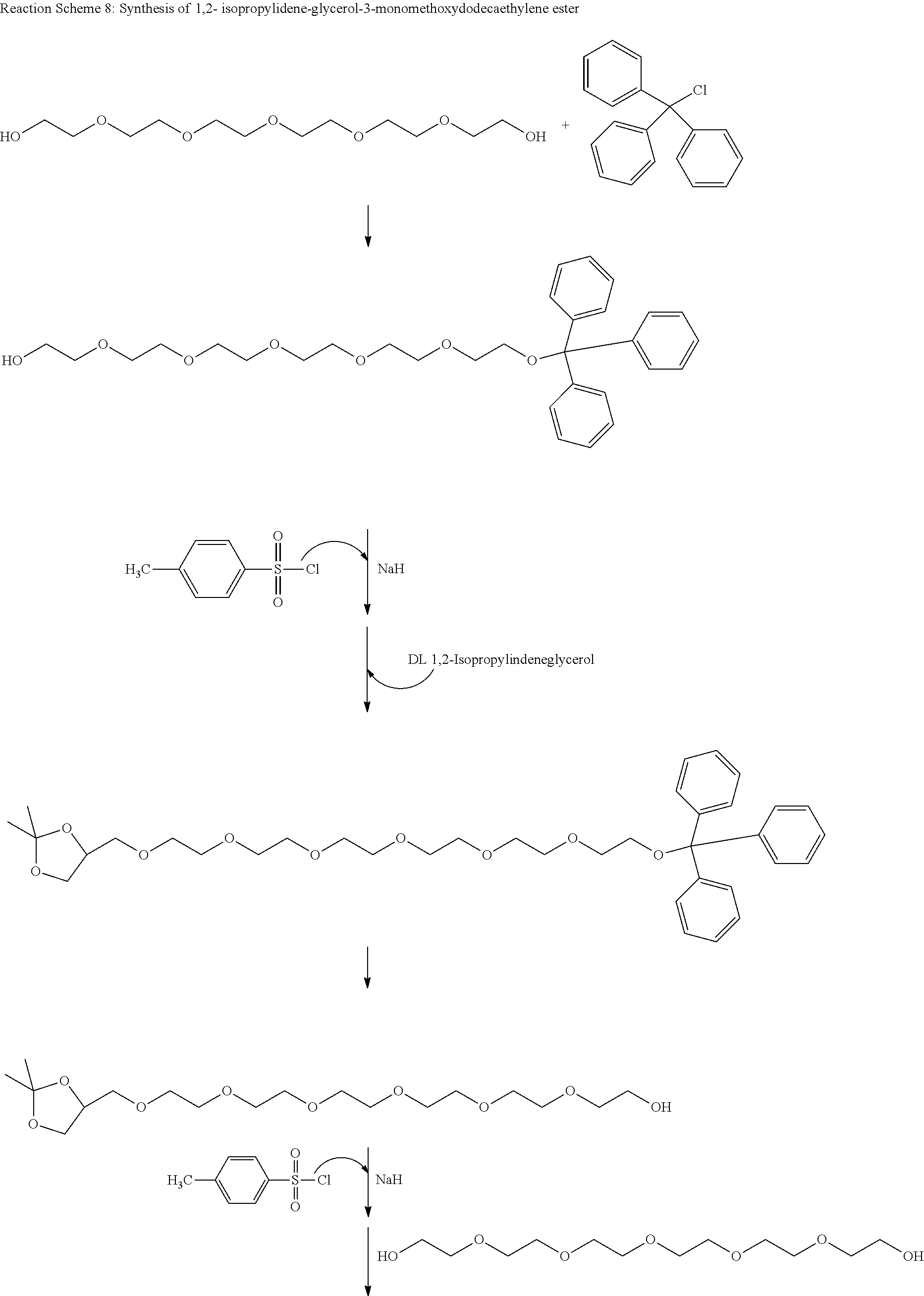

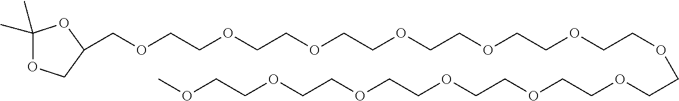

1 moles of hexaethylene glycol was mixed with 0.15 moles of pyridine and heated to 45-50° C. and 0.1 moles of trityl chloride was added. The reaction was carried over night (approximately 16 hours) under constant stirring and then cooled down to room temperature and extracted with toluene. The extract was washed with water, then extracted with hexane and dried over MgSO₄. The solvent was removed under vacuum, a light yellow oily Tr-hexaethylene glycol was obtained (yield 70 to 85%).

0.1 moles of Tr-hexaethylene glycol and 0.101 moles of p-toluenesulfonyl chloride were mixed in 100 mL of methylene chloride. The homogeneous mixture was cooled to 0° C. in a dry-ice-acetone bath and 45 g of KOH was added in small portions under vigorous stirring while maintaining the reaction temperature below 5° C. The reaction was completed under constant stirring for 3 hours at 0° C. The crude product was diluted with 100 mL of methylene chloride, then 120 mL of ice-cold water was added. The organic layer was collected, and the aqueous phase was extracted with methylene chloride (2×50 mL). The combined organic layers were washed with water (100 mL) and dried over MgSO₄. The solvent was removed under vacuum to yield (87 to 99%) clear oil.

To a three-necked flask, 1.2-isopropylidene-rac-glycerol (0.1 mol) and NaH (0.4 mol) and dry THF (200 mL) were charged. A dry THF solution (125 mL) of Tr-hexaethylene glycol tosylate (0.1 mol) was added to the mixture dropwise at room temperature. The mixture was refluxed for 24 hours, and cooled to room temperature. Ice-cold methanol was added to the reaction mixture to quench excessive NaH. The solvent was evaporated and the crude product was extracted with 5% HCl (w/v) and CH₂Cl₂. The crude product was not purified further but taken directly to the next stage of synthesis.

The above crude product was transferred to a high pressure resistant glass flask and 200 mL of dry methylene chloride and 10% palladium on carbon (1.5 g). Hydrogenolysis was carried out by purging pure hydrogen at 30° C. in atmosphere for approximately 60 minutes to remove the protective group on the hexaethylene glycol. After the hydrogen was replaced by nitrogen, the solution was cooled to 4 to 6° C. and the catalyst was removed by filtration. Solvent was evaporated to yield 95 to 98% of the final product.

In a three-necked flask, 3-hexaethylene-glycol-1,2-isopropylidene-rac-glycerol (0.1 mol) and NaH (0.4 mol) and dry THF (500 mL) were mixed. A dry THF solution (200 mL) of monobenzylhexaethylene glycol tosylate (0.11 mmol) was added to the mixture dropwise at room temperature. The mixture was refluxed for 24 hours, and then cooled to room temperature. Ice-cold methanol was added to the reaction mixture to quench excessive NaH. The solvent was evaporated and the crude product was extracted with 5% HCl (w/v) and CH₂Cl₂. The solvent was evaporated and further purified by gel permeation chromatography to yield 82% of 3-monomethoxydodecaethylene glycol-1,2-isopropylideneglycerol.

The isopropylidene protecting group was removed by stirring 10 g of 3-monomethoxydodecaethylene glycol-1,2-Isopropylideneglycerol for 3 hours in acidic methanol solution (180 mL MeOH:20 mL, 1 M HCl). The mixture was neutralized with sodium hydrogen carbonate and extracted in chloroform (3×150 mL) and dried over sodium sulfate. Filtration and evaporation of the solvent yields the product (75-80%) of 3-monomethoxydodecaethylene glycol-1,2-dihydroxyl-glycerol (Chemical Structure 16).

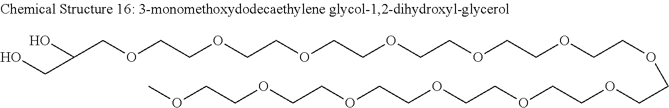

In the above PEG chain extension reaction, the starting PEG reagent preferably comprises 1 to 6 CH₂CH₂O unit, and more preferably has 3 to 6 CH₂CH₂O unit, and more preferably has 4 to 6 CH₂CH₂O units. The reaction between glycerol and the PEG-reagent can occur in the presence or the absence of a linker group. Preferred PEG-reagents have hydroxyl, amino, carboxyl, isocyanate, thiol, carbonate functional groups. Especially preferred PEG-reagents for use in this embodiment of the inventive method include PEG-tosylate, PEG-mesylate and succinyl-PEG. Following the reaction between the glycerol and the PEG-reagent, the protecting groups are removed.

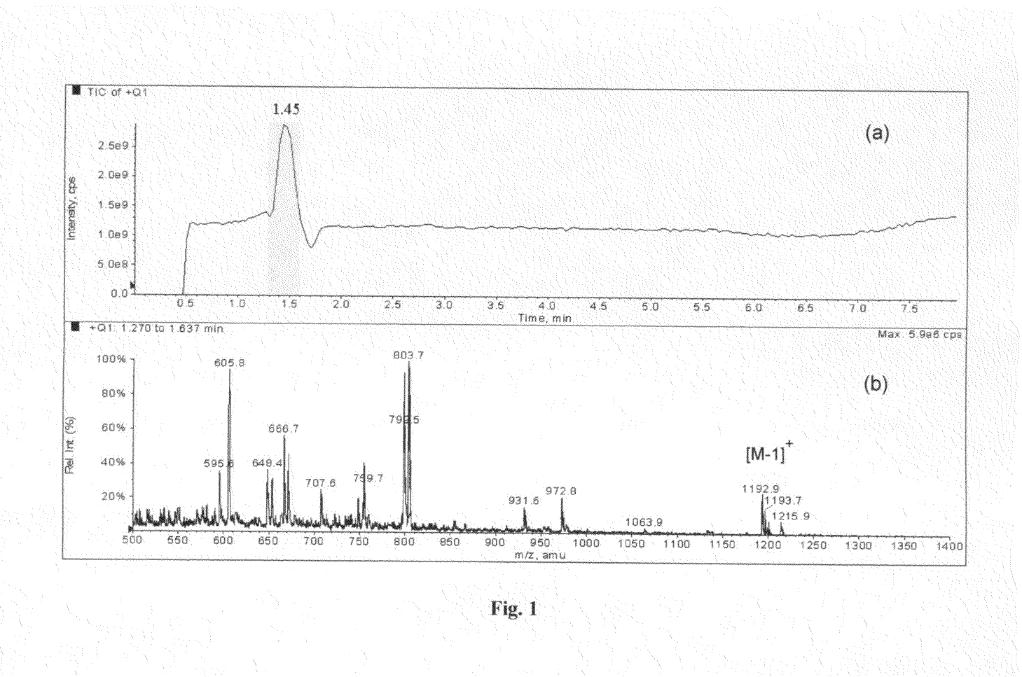

0.1 moles of 3-monomethoxydodecaethylene glycol-1,2-dihydroxyl-glycerol was constantly stirred under nitrogen in 250 mL of chloroform. 0.21 mole of oleoyl chloride was dissolved with 250 mL of chloroform and added to this heterogeneous mixture of dihydroxyacetone and followed by adding 15 mL of anhydrous pyridine. The reaction proceeded for 30 minutes under constantly stirring at room temperature. The mixture turned homogeneous and the reaction was completed when no detectable oleoyl chloride was in the mixture. The bulk solvent was removed under vacuum. The residue was diluted with methylene chloride and equal volume of brine solution was added. The organic layer was collected and the aqueous phase was repeatedly extracted with methylene chloride and the organic layers were combined and washed again with water (50 mL) and dried over sodium sulfate, and further evaporated to yield a (70-80%) oily product (Chemical Structure 17). Its liquid chromatograph-mass spectrometry (LC-MS) chromatogram is shown in FIG. 1: (a) the sample was injected onto a 4.6×50 mm Inertsil C8 column and eluted under a mixture of Tetrahydrofuran and H₂O (4/6, v/v) monitoring with a mass spectrometry and (b) the MS spectrum of the peak eluted at 1.45 minutes where [M-1]⁺ is the ion of the parent compound.

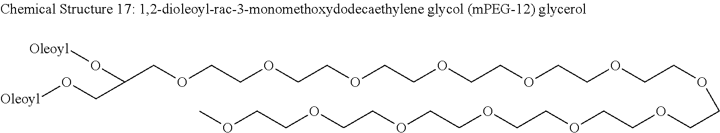

### Example 3

### Synthesis of 1,3-dioleoyl-rac-2-monomethoxyDodecaethylene glycol (mPEG-12)-glycerol

The general steps for this synthesis is showed in the following scheme (Reaction Scheme 9):

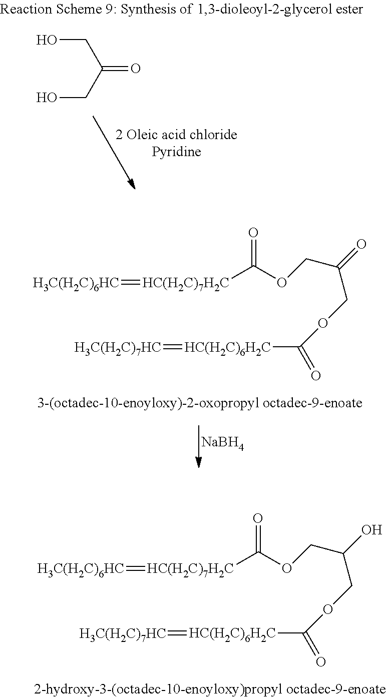

0.033 moles of dihydroxyacetone was constantly stirred under nitrogen in 150 mL of chloroform. 0.06 mole of oleoyl chloride was dissolved with 150 mL of chloroform and added to this heterogeneous mixture of dihydroxyacetone and followed by adding 10 mL of anhydrous pyridine. The reaction proceeded for 30 minutes under constant stirring at room temperature. The mixture turned homogeneous and the reaction was completed when no detectable oleoyl chloride was in the mixture. The bulk solvent was removed under vacuum. The residue was wash with water then extracted with ethyl acetate. The aqueous phase was repeatedly extracted with ethyl acetate and the organic layers were combined and washed again with water, dried over sodium sulfate and evaporated. The resulting oily product was recrystallized from methanol to give 3-(octadec-10-enoyloxy)-2-oxopropyl octadec-9-enoate (% of yields 75-80) with a melting temperature of 43-44°.

The 1,3-dioleate (0.02 moles) was dissolved with 150 mL of tetrahydrofuran (THF) and 10 mL of water. The heterogeneous solution was chilled to 5° C. in an ice-bath. A solution of sodium borohydride (0.026 mol in THF) was added in small portions. After 30 minutes excess borohydride was destroyed by adding approximately 1 mL of glacial acetic acid, the solution was then diluted with chloroform, and washed with water and dried over magnesium sulfate. An oil was obtained which partially crystallized to needle-like crystals of 2-hydroxy-3-(octadec-10-enoyloxy)propyl octadec-9-enoate (yields 80 to 90%) with a melting temperature of 20-22° C.

From the above intermediate product, 1,3-dioleoyl-rac-glycerol-rac-2-monomethoxy-dodecaethylene glycol (mPEG-12)-glycerol (Chemical Structure 18) was prepared after the reaction and work-up as described in the Examples 1 and 2.

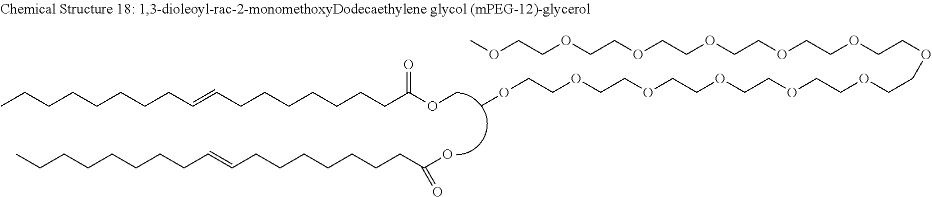

### Example 4

### 1,2-dimyristoyl-rac-3-dodecapropylene glycol (PPG-12)-glycerol

The general steps for this synthesis is showed in the following scheme (Reaction Scheme 10):

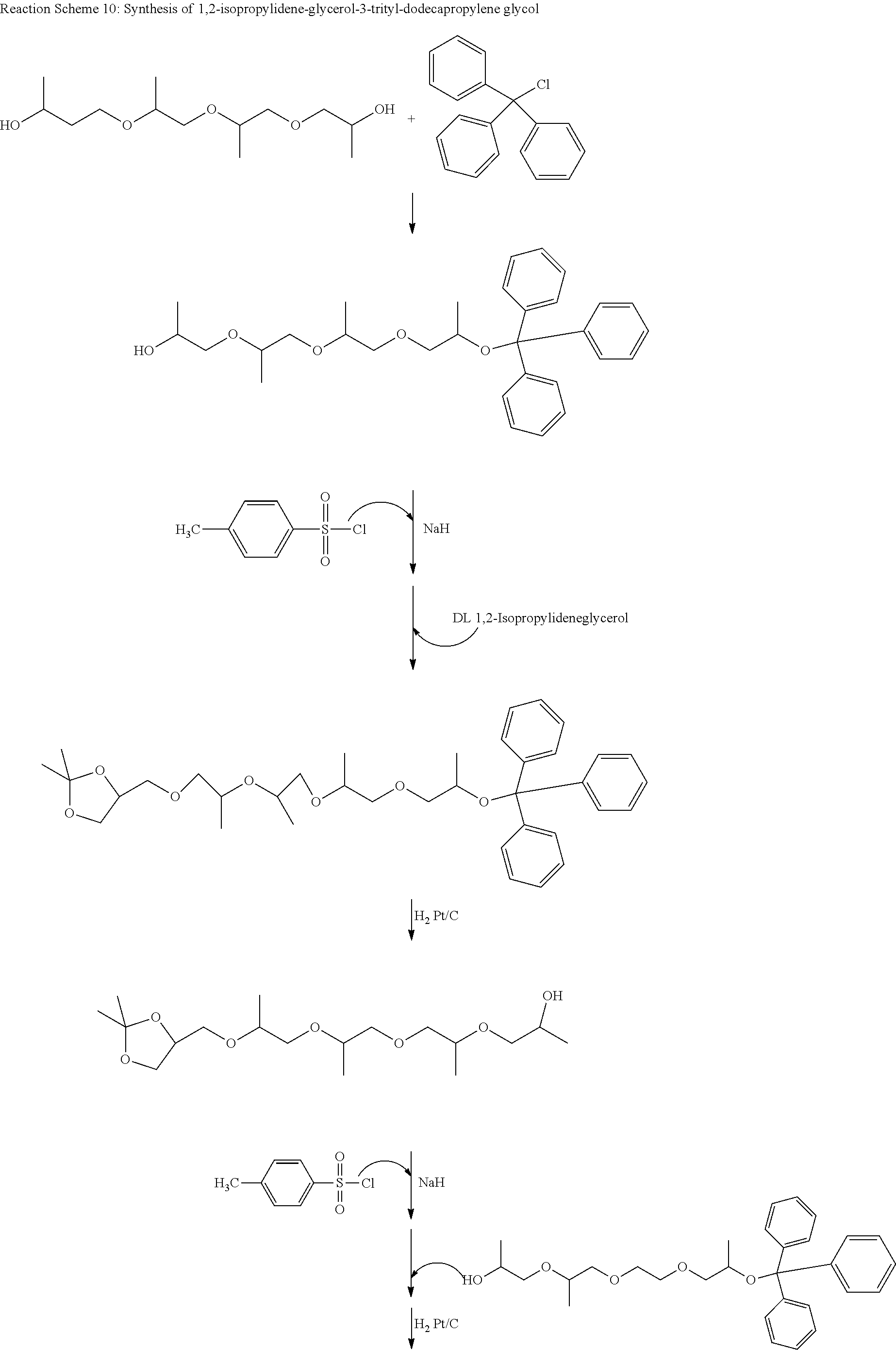

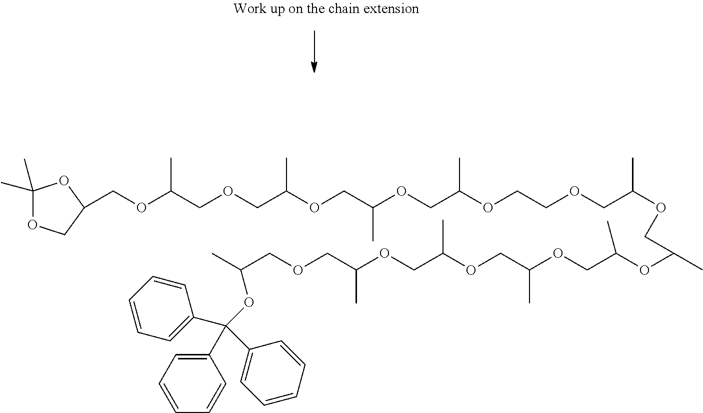

1.5 moles of tetrapropylene glycol was mixed with 0.23 moles of pyridine and heated to 45-50° C. and 0.15 moles of trityl chloride was added. The reaction was carried over night (approximately 16 hours) under constant stirring, then cooled down to room temperature and extracted with toluene. The extract was washed with water, then extracted with hexane and dried over MgSO₄. The solvent was removed under vacuum. A light yellow oily Tr-tetrapropylene glycol was obtained (yield 75 to 85%).

0.1 moles of Tr-tetrapropylene glycol and 0.101 moles of p-toluenesulfonyl chloride were mixed in 100 mL of methylene chloride. The homogeneous mixture was cooled to 0° C. in a dry-ice-acetone bath and 45 g of KOH was added in small portions under vigorous stirring while maintaining the reaction temperature below 5° C. The reaction was completed under constant stirring for 4 hours at 0° C. The crude product was diluted with 100 mL of methylene chloride, then 120 mL of ice-cold water was added. The organic layer was collected, and the aqueous phase was extracted with methylene chloride (2×50 mL) The combined organic layers were washed with water (100 mL) and dried over MgSO₄. The solvent was removed under vacuum to yield (85 to 95%) clear oil.

To a three-necked flask, 1,2-isopropylidene-rac-glycerol (0.1 mol) and NaH (0.4 mol) and dry THF (200 mL) were charged. A dry THF solution (125 mL) of Tr-tetrapropylene glycol tosylate (0.1 mol) was added to the mixture dropwise at room temperature. The mixture was refluxed for 24 hours and then cooled to room temperature. Ice-cold methanol was added to the reaction mixture to quench excessive NaH. The solvent was evaporated and the crude product was extracted with 5% HCl (w/v) and CH₂Cl₂. The crude product was not purified further but taken directly to the next stage of synthesis.

The above crude product was transferred to a high pressure resistant glass flask and 200 mL of dry methylene chloride and 10% palladium on carbon (1.5 g). Hydrogenolysis was carried out by purging pure hydrogen at 30° C. in atmosphere for approximately 60 minutes to remove the protective group on the hexaethylene glycol. After the hydrogen was replaced by nitrogen, the solution was cooled to 4 to 6° C. and the catalyst was removed by filtration. Solvent was evaporated to yield 95 to 98% of the final product.

To a three-necked flask, 3-tetrapropylene-glycol-1.2-isopropylidene-rac-glycerol (0.1 mol) and NaH (0.4 mol) and dry THF (500 mL) were added. A dry THF solution (200 mL) of Tr-tetrapropylene glycol tosylate (0.11 mmol) was added to the mixture dropwise at room temperature. The mixture was refluxed for 24 hours, and cooled to room temperature. Ice-cold methanol was added to the reaction mixture to quench excessive NaH. The solvent was evaporated and the crude product was extracted with 5% HCl (w/v) and CH₂Cl₂.

The above etherification steps were repeated one more time. The solvent was evaporated and further purified by gel permeation chromatography to yield approximately 80% of 3-trityl-dodecapropylene glycol-1,2-isopropylideneglycerol.

The isopropylidene protecting group was removed by stirring 10 g of 3-dodecapropylene glycol-1,2-isopropylideneglycerol for 3 hours in acidic methanol solution (180 mL MeOH:20 mL, 1 M HCl). The mixture was neutralized with sodium hydrogen carbonate and extracted in to chloroform (3×150 mL) and dried over sodium sulfate. Filtration and evaporation of the solvent yielded the product (75-80%) of 3-trityl-dodecapropylene glycol-1,2-dihydroxyl-glycerol (Chemical Structure 19).

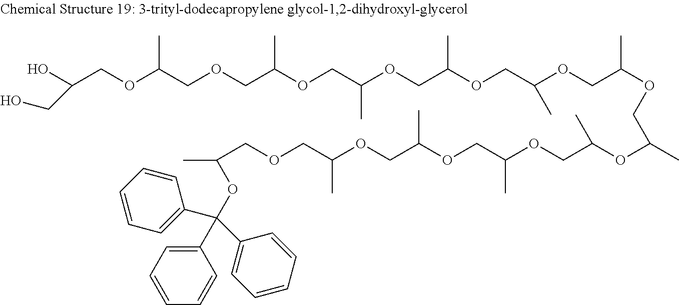

In the above PEG chain extension reaction, the starting PEG reagents preferably comprise 1 to 6 CH₂(CH₃)CH₂O units, and more preferably 3 to 6 CH₂CH₂O units, and more preferably has 4 to 6 CH₂CH₂O units. The reaction between glycerol and the PEG-reagent can occur in the presence or the absence of a linker group. In this embodiment, preferred PEG-reagents have hydroxyl, amino, carboxyl, isocyanate, thiol, carbonate functional groups. Especially preferred PEG-reagents for use in this embodiment of the inventive method include PEG-tosylate, PEG-mesylate and succinyl-PEG. Following the reaction between the glycerol and the PEG-reagent, the protecting groups are removed.

0.1 moles of 3-trityledodecapropylene glycol-1,2-dihydroxyl-glycerol was constantly stirred under nitrogen in 250 mL of chloroform. 0.21 mole of myristic chloride was dissolved with 250 mL of chloroform and added to this heterogeneous mixture of dihydroxyacetone and followed by adding 15 mL of anhydrous pyridine. The reaction proceeded for 30 minutes under constant stirring at room temperature. The mixture turned homogeneous and the reaction was completed when no detectable oleoyl chloride was in the mixture. The bulk solvent was removed under vacuum and transferred to next step without further purification.

The above crude product was transferred to a high pressure resistant glass flask and 200 mL of dry methylene chloride and 10% palladium on carbon (1.5 g). Hydrogenolysis was carried out by purging pure hydrogen at 30° C. in atmosphere for approximately 60 minutes to remove the protective group on the hexaethylene glycol. After the hydrogen was replaced by nitrogen, the solution was cooled to 4 to 6° C. and the catalyst was removed by filtration. Solvent was evaporated to yield 95 to 98% of the final product.

The residue from the above was diluted with methylene chloride and equal volume of brine solution was added. The organic layer was collected and the aqueous phase was repeatedly extracted with methylene chloride and the organic layers were combined and washed again with water (50 mL) and dried over sodium sulfate, and further evaporated to yield a (70-85%) oily product (Chemical Structure 20).

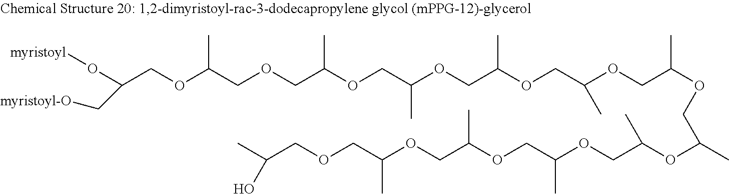

For instance, the starting reagents in the polymer chain extension reaction, can be methylene glycol or ethylene glycol or propylene glycol or a mixture of the three from 1 to 6 repeating unit, and more preferably has 3 to 6 repeating unit, and more preferably has 4 to 6 repeating unit. The reaction between glycerol and the reagent can occur in the presence or the absence of a linker group. In this embodiment, preferred polymerization reagents have hydroxyl, amino, carboxyl, thiol, isocyanate, carbonate functional groups. Especially the preferred reagents for use in this embodiment of the inventive method include tosylate, mesylate and succinyl activated intermediates. Following the reaction between the glycerol and the polymerization-reagent, the protecting groups are removed. One of such examples is as showed in Chemical Structure 21.

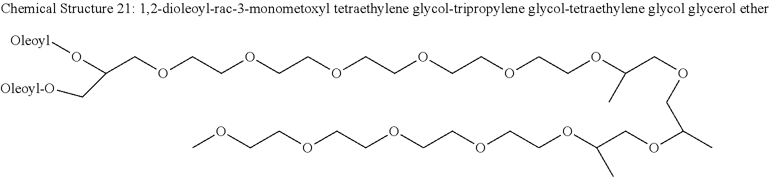

### Example 5

### Solid Dose Compositions

A liquid PEG-lipid conjugate is added to a stainless steel vessel equipped with propeller type mixing blades. The drug substance is added with constant mixing. Mixing continues until the drug is visually dispersed in the lipids at a temperature to 55°-65° C. In a separate container, a PEG-lipid conjugate with a melting temperature above about 30 degrees C. is melted with heating or dissolved in ethanol and added to the vessel with mixing. Mixing continues until fully a homogenous solution is achieved. If necessary, ethanol is removed by vacuum. The solution is filled into capsule shells or predesigned packaging configurations (molds) when the solution is warm. Filled capsules or molds are placed under refrigeration (2-8° C.) until the cream-like mixture is solidified when cooled. A sample formulation is described in Table 5.

TABLE 5

| Ingredient | % |
|---|---|
| Drug Substance | 15 |
| Liquid PEG-lipid Conjugate | 40 |
| Solid PEG-lipid Conjugate | 45 |
| Ethanol | <1 |

The liquid conjugate may be GDM-12, GDO-12, GDC-12, GDM-600, GDO-600, GDC-600, GOB-12, GMB-12, GOBH, GMBH, GCBH, GCBH or GPBH. The solid lipid conjugate may be GDS-12, DSB-12, GDO-23, GDO-27, GDM-23, GDM-27 and GDS-23. The drug may be modafinil or nifedapine or esomeprazole or rapamycin or another active agent.

### Example 6

### Solid Dose Compositions

A liquid PEG lipid conjugate (having a melting point below about 15 degrees C.) was added to a stainless steel vessel equipped with propeller type mixing blades. The drug substance was added with constant mixing. Mixing continued until the drug was visually dispersed in the lipids at a temperature to 55°-65° C. In a separate container, TPGS-VE was dissolved in ethanol and added to the vessel with mixing. Mixing continued until fully a homogenous solution was achieved. Ethanol was be removed by vacuum. The solution was filled into capsule shells or predesigned packaging configuration (molds) when the solution was warm. The filled capsules or molds were placed under refrigeration (2-8° C.). The cream-like mixture was solidified when cooled. A sample formulation is described in Table 6.

TABLE 6

| Ingredient | % |
|---|---|
| Drug Substance (active) | 15 |
| Lipid PEG-lipid Conjugate | 40 |
| TPGS-VE | 45 |
| Ethanol | <1 |

The liquid conjugate may be GDM-12, GDO-12, GDC-12, GDM-600, GDO-600, GDC-600, GOB-12, GMB-12, GOBH, GMBH, GCBH, GCBH or GPBH. The drug may be modafinil or nifedapine or esomeprazole or rapamycin or another active agent.

### Example 7

### Oral Solution Compositions

PEG-lipid was added to a vessel equipped with a mixer propeller. The drug substance was added with constant mixing. Mixing continued until the drug was visually dispersed in the lipids. Pre-dissolved excipients were slowly added to the vessel with adequate mixing. Mixing continued until fully a homogenous solution was achieved. A sample formulation is described in Table 7.

TABLE 7

| Ingredient | mg/mL |
|---|---|
| Drug Substance (active) | 30.0 |
| PEG Lipid | 100 |
| Lactic Acid | 50 |
| Sodium Hydroxide | See below |
| Hydrochloric Acid | See below |
| Sodium Benzoate | 2.0 |
| Artificial Flavor | 5.0 |
| Purified Water | qs 1 mL |

The lipid may be GDM-12, GDO-12, GDC-12, GDM-600, GDO-600, GDC-600, GOB-12, GMB-12, GOBH, GMBH, GCBH, GCBH or GPBH or any combination thereof. Sodium hydroxide is used to prepare a 10% w/w solution in purified water. The targeted pH is in a range of 4.0 to 7.0. NaOH is used to adjust pH if necessary. The drug may be modafinil or nifedapine or esomeprazole or rapamycin or another active agent.

### Example 8

### Cyclosporine Ophthalmic Compositions

PEG-lipid was added to a vessel equipped with a mixer propeller. The cyclosporine drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. A sample formulation is described in Table 8.

TABLE 8

| Ingredient | mg/100 mL |
|---|---|
| Cyclosporine | 50 mg |
| PEG Lipid | 500 |
| Sodium Hydroxide | See below |
| Hydrochloric Acid | See below |
| Sodium Chloride | 900 |
| Sterile purified water | qs 100 mL |

The lipid may be GDM-12, GDO-12, GDC-12, GDM-600, GDO-600, GDC-600, GOB-12, GMB-12, GOBH, GMBH, GCBH, GCBH or GPBH or thereof. Sodium hydroxide is used to prepare a 10% w/w solution in purified water. The targeted pH is in a range of 6.0 to 7.4. NaOH is used to adjust pH if necessary.

### Example 9

### Injection Solution Compositions

The injectable solution was prepared as in Example 7, except that the targeted pH range was between 6.0 and 8.0. A sample formulation is described in Table 9.

TABLE 9

| Ingredient | mg/mL |
|---|---|
| Drug Substance (Active) | 10.0 |
| PEG Lipid | 100 |
| Sodium Hydroxide | See Below |
| Lactic Acid | 20 |
| Purified Water | qs 1 mL |

The lipid may be GDM-12, GDO-12, GDC-12, GDM-600, GDO-600, GDC-600, GOB-12, GMB-12, GOBH, GMBH, GCBH, GCBH or GPBH or any combination thereof. Sodium hydroxide is used to prepare a 10% w/w solution in purified water. The targeted pH is in a range of 6.5 to 7.4. NaOH is used to adjust pH if necessary. The drug may be triazoles including posaconazole, voriconazole and itraconazole or rapamycin or cyclosporines or tacrolimus or or nifedipine or paclitaxel or docetaxel or gefitinib or propofol or rifampin or diazepam or nelfinavir or another active agent.

### Example 10

### Pharmacokinetic Profile and Bioavailability of Itraconazole Formulations

Groups of three male mice (B6D2F1) were used for the studies. Pharmacokinetics (PK) were performed on heparinized mouse plasma samples obtained typically at 0 hr, 0.08 hr, 0.25 hr, 0.5 hr, 1 hr, 2 hr, 4 hr, 8 hr, 16 hr and 24 hr after the bolus IV injection or oral feeding at 0 hr, 0.5 hr, 1 hr, 2 hr, 4 hr, 8 hr, 16 hr and 24 hr for itraconazole. Samples were analyzed using a HPLC-MS/MS method. To determine the level of each drug, the drug was first isolated from plasma with a sample pre-treatment. Acetonitrile were used to remove proteins in samples. An isocratic HPLC-MS/MS method was then used to separate the drugs from any potential interference. Drug levels were measured by MS detection with a multiple reaction monitoring (MRM) mode. PK data was analyzed using the WinNonlin program (ver. 5.2, Pharsight) compartmental models of analysis.

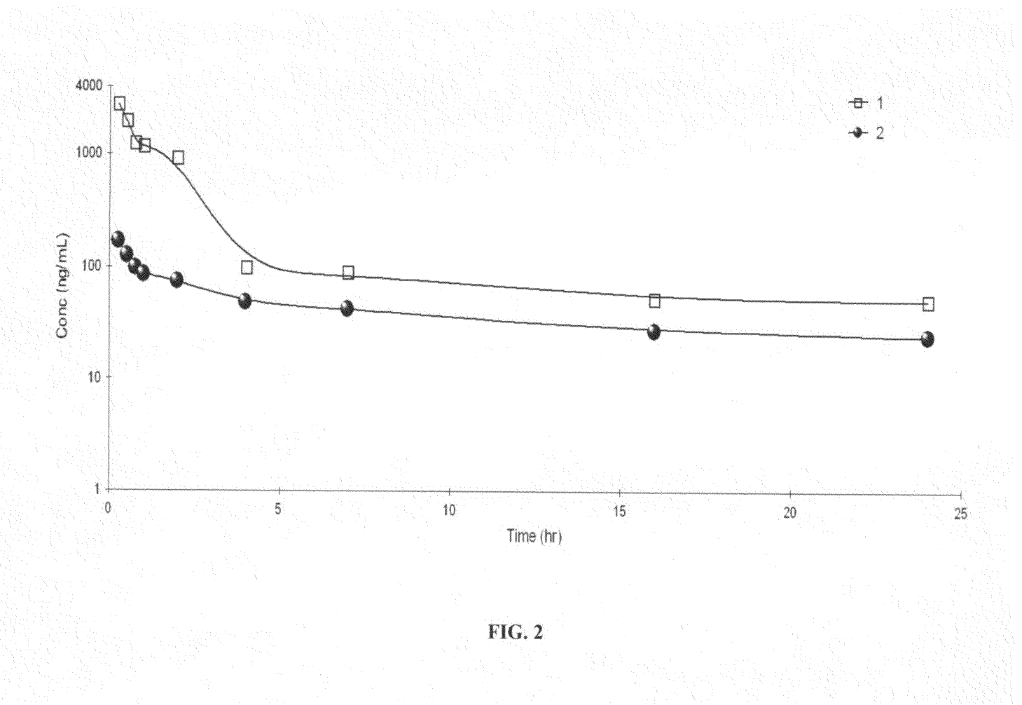

FIG. 2 shows mouse PK profiles of itraconazole formulations with (1) GDO-12 (1:10 drug to lipid ratio) in 10 mM of sodium phosphate buffer (pH 7.4) and (2) 10% Cremophor-5% MeOH in 10 mM of sodium phosphate buffer (pH 7.4). The drug was administered intravenously and the dosing strength was 20 mg/kg. The AUC were 5441 μg·hr/mL and 986 μg·hr/mL for the DAG-PEG formulation (1) and the commercial product (2), respectively.

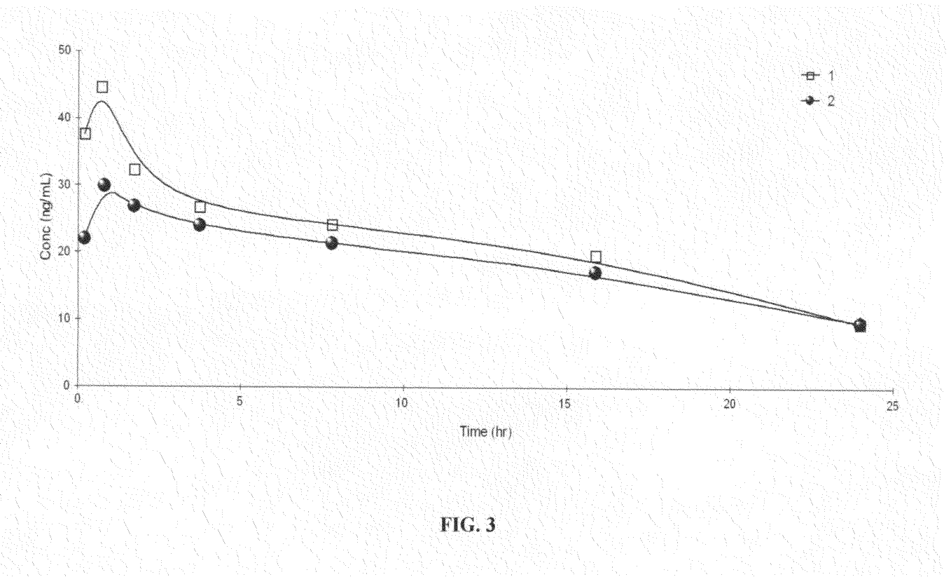

FIG. 3 shows mouse PK profiles of itraconazole formulations with (1) GDO-12 (1:10, drug to lipid ratio) in 10 mM of sodium phosphate buffer (pH 7.4) and (2) 10% Cremophor-5% MeOH in 10 mM of sodium phosphate buffer (pH 7.4). The drug was administered orally and the dosing strength was 20 mg/kg. The relative bioavailability (based on the AUC_{0-24 hr}) were 63% and 45% for the formulations of PEG-DAG (1) and (2), respectively.

### Example 11

### Topical Cream Composition

PEG lipid was added to a stainless steel vessel equipped with propeller type mixing blades. The drug substance was added with constant mixing. Mixing continued until the drug was visually dispersed in the lipids at a temperature to 60°-65° C. Organic acid, Cholesterol and glycerin were added with mixing. Ethanol and ethyoxydiglycol were added with mixing. Finally Carbopol ETD 2020, purified water and triethylamine were added with mixing. Mixing continued until fully a homogenous cream was achieved. The formulation is described in Table 10.

TABLE 10

| Ingredient | % |
|---|---|
| Drug Substance (Active) | 1.0 |
| PEG Lipid | 5.0 |
| Carbopol ETD 2020 | 0.5 |
| Ethyoxydiglycol | 1.0 |
| Ethanol | 5.0 |
| Glycerin | 1.0 |
| Cholesterol | 0.4 |
| Triethylamine | 0.20 |
| Organic acid | 10 |
| Sodium hydroxide | See below |
| Purified water | qs 100 |

The lipid may be GDM-12, GDO-12, GDC-12, GDM-600, GDO-600, GDC-600, GOB-12, GMB-12, GOBH, GMBH, GCBH, GCBH or GPBH or GDS-12 or any combination thereof. Organic acid may be lactic acid or pyruvic acid or glycolic acid. Sodium hydroxide is used to adjust pH if necessary. The targeted pH range was between 3.5 and 7.0. The drug may be itraconazole, posaconazole, voriconazole or equaconazole, Terbinafine, Amorolfine, Naftifine, Butenafine, Benzoic acid, Ciclopirox, Tolnaftate, Undecylenic acid, Flucytosine, Griseofulvin, Haloprogin, Sodium bicarbonate or Fluocinolone acetonide.

### Example 12

### Topical Solution Composition

The topical solution was prepared as in Example 11, a sample formulation is described in Table 11.

TABLE 11

| Ingredient | % |
|---|---|
| Drug Substance (Active) | 1.0 |
| PEG Lipid | 5.0 |
| α-Tocopherol | 0.5 |
| Organic acid | 10.0 |
| Ethanol | 5.0 |
| Sodium Benzoate | 0.2 |
| Sodium Hydroxide | See Below |
| Purified Water | qs 100 |

The lipid may be GDM-12, GDO-12, GDC-12, GDM-600, GDO-600, GDC-600, GOB-12, GMB-12, GOBH, GMBH, GCBH, GCBH or GPBH or any combination thereof. Organic acid may be lactic acid or pyruvic acid or glycolic acid. Sodium hydroxide is used to adjust pH if necessary. The targeted pH range was between 3.5 and 7.0. The drug may be itraconazole, posaconazole, voriconazole or equaconazole, Terbinafine, Amorolfine, Naftifine, Butenafine, Benzoic acid, Ciclopirox, Tolnaftate, Undecylenic acid, Flucytosine, Griseofulvin, Haloprogin, Sodium bicarbonate or Fluocinolone acetonide.

### Example 13

### Azithromycin Ophthalmic Compositions

PEG-lipid was added to a vessel equipped with a mixer propeller. The azithromycin drug substance was added with constant mixing. Mixing continued until the drug was visually dispersed in the lipids. Pre-dissolved excipients and sterile purified water were slowly added to the vessel with adequate mixing. Mixing continued until fully a homogenous solution was achieved. A sample formulation is described in Table 12.

TABLE 12

| Ingredient | mg/mL |
|---|---|
| Azithromycin | 15 mg |
| PEG Lipid | 150 |
| Sodium Hydroxide | See below |
| Hydrochloric Acid | See below |
| Sodium Chloride | 9 |
| Sterile purified water | qs 1 mL |

The lipid may be GDM-12, GDO-12, GDC-12, GDM-600, GDO-600, GDC-600, GOB-12, GMB-12, GOBH, GMBH, GCBH, GCBH or GPBH or any combination thereof. Sodium hydroxide is used to prepare a 10% w/w solution in purified water. The targeted pH is in a range of 7.0 to 7.8. NaOH is used to adjust pH if necessary.

Preferable concentration of Azithromycin is 0.5 to 3%, more preferable is 0.5 to 2%, most preferable is 1 to 2%. The preferable ratio of PEG-lipid to the drug (PEG-Lipid/cyclosporine) is 1 to 20, more preferable is 3 to 15, most preferable is 5 to 10.

We claim:

1. A method of making a PEG chain of a defined length, the method comprising:
(a) selecting a glycerol derivative with a glycerol protecting group that is stable under a first set of conditions and convertible to free hydroxyl groups under a second set of conditions;
(b) selecting a initial PEG oligomer having between 1 and 12 subunits, where the initial PEG oligomer has an oligomer protecting group on its first terminus and the said oligomer protecting group converts to a hydroxyl group under the first set of conditions, and where the initial PEG oligomer has a reactive group on its second terminus, said reactive group forming a bond with a compound having a free hydroxyl group;
(c) reacting the glycerol derivative with the initial PEG oligomer to form a glycerol-PEG conjugate;
(d) removing the oligomer protective group by exposing the conjugate to the first set of conditions;
(e) repeating steps (f), (g) and (h) between 0 and 6 additional times, where steps are as described below;
(f) selecting an extending PEG oligomer having between 2 and 11 subunits, where the extending PEG oligomer has an oligomer protecting group on its first terminus and the said oligomer protecting group converts to a hydroxyl group under the first set of conditions, and where the extending PEG oligomer has a reactive group on its second terminus, said reactive group forming a bond with a compound having a free hydroxyl group;
(g) reacting the glycerol-PEG conjugate with the extending PEG oligomer to form an extended glycerol-PEG conjugate;
(h) removing the oligomer protective group by exposing the conjugate to the first set of conditions;
(i) terminating the PEG chain by either step (j) or steps (k) and (l), where the steps are as described below;
(j) adding a terminal group to the free hydroxyl group of the extended glycerol-PEG conjugate; or
(k) selecting a terminal PEG oligomer having between 2 and 11 subunits, where the terminal PEG oligomer has terminal group on its first terminus, and where the terminal PEG oligomer has a reactive group on its second terminus, said reactive group forming a bond with a compound having a free hydroxyl group; and
(l) reacting the glycerol-PEG conjugate or extended glycerol-PEG conjugate with the terminal PEG oligomer; and
(m) exposing the terminated glycerol-PEG conjugate to the second set of conditions.

2. The method of claim 1, where the terminal group is methyl.

3. The method of claim 1, where the first set of conditions is catalytic reduction.

4. The method of claim 1, where the second set of conditions is exposure to acid.

5. The method of claim 1, where the glycerol derivative is a compound represented by the formula shown at Reaction Scheme 1(a).

6. The method of claim 1, where the glycerol derivative is a compound represented by the formula: Chemical Structure 2.

7. The method of claim 1, where the glycerol derivative is a compound represented by the formula: Chemical Structure 3.

8. The method of claim 1, where the glycerol derivative is a compound represented by the formula: Chemical Structure 4.

9. The method of claim 1, where the glycerol protecting group is an alkyl group.

10. The method of claim 1, further comprising the steps of:
(n) removing the glycerol protecting group; and
(o) bonding a lipid group to the glycerol backbone.

11-76. (canceled)
